(19) United States
(12) Patent Application Publication
(10) Pub. No.: US 2025/0188529 A1
(43) Pub. Date: Jun. 12, 2025

(54) CAPTURING AND AMPLIFYING POLYNUCLEOTIDES USING MOLECULES AND PARTICLES

(71) Applicant: Illumina, Inc., San Diego, CA (US)

(72) Inventors: Xavier von Hatten, Cambridge (GB); Gianluca Artioli, Cambridge (GB); Nam Nguyen, Cambridge (GB); Jonathan Boutell, Bishops Stortford (GB)

(73) Assignee: Illumina, Inc., San Diego, CA (US)

(21) Appl. No.: 18/977,460

(22) Filed: Dec. 11, 2024

Related U.S. Application Data
(60) Provisional application No. 63/609,211, filed on Dec. 12, 2023.

(51) Int. Cl. C12Q 1/6844; B01L 3/00; C12Q 1/6804

(52) U.S. Cl. CPC C12Q 1/6844; B01L 3/502; C12Q 1/6804; B01L 2200/0647; B01L 2300/0829

(57) ABSTRACT

In some examples, a device includes a flowcell including wells, and a plurality of molecules. Each molecule may include a single respective polynucleotide. At least some of the wells are coupled to a single respective one of the molecules such that a single respective polynucleotide is coupled to those wells. The device also may include a plurality of particles. Each particle may include amplification primers and may be coupled to a single one of the wells via hybridization between an amplification primer of that particle and the polynucleotide of the molecule coupled to that well.

### CROSS-REFERENCE TO RELATED APPLICATIONS

This application claims the benefit of U.S. Provisional Patent Application No. 63/609,211, filed Dec. 12, 2023 and entitled “Capturing and Amplifying Polynucleotides Using Molecules and Particles,” the entire contents of which are incorporated by reference herein.

### INCORPORATION BY REFERENCE OF SEQUENCE LISTING

The material in the accompanying sequence listing is hereby incorporated by reference into the application. The accompanying sequence listing XML file, named “IP-2727-US.xml”, was created on Nov. 20, 2024 and is 28 kB in size.

### FIELD

This application generally relates to capturing and amplifying polynucleotides.

### BACKGROUND

Cluster amplification is an approach to amplifying polynucleotides, for example for use in genetic sequencing. Target polynucleotides are captured by primers (e.g., P5 and P7 primers) coupled to a substrate surface in a flow cell, and form “seeds” at random locations on the surface. Cycles of amplification are performed to form clusters on the surface around each seed. The clusters include copies, and complementary copies, of the seed polynucleotides. In some circumstances, the substrate is patterned so as to define regions that bound different clusters, such as wells that may be filled with respective clusters.

### SUMMARY

Examples provided herein are related to capturing and amplifying polynucleotides using molecules and particles.

Some examples herein provide a device. The device may include a flowcell including wells. The device may include a plurality of molecules. Each molecule may include a single respective polynucleotide. At least some of the wells are coupled to a single respective one of the molecules such that a single respective polynucleotide is coupled to those wells. The device may include a plurality of particles. Each particle may include amplification primers and may be coupled to a single one of the wells via hybridization between an amplification primer of that particle and the polynucleotide of the molecule coupled to that well.

In some examples, each of the molecules includes: a dendritic core to which the single respective polynucleotide is coupled; and a plurality of dendrons. Each of the dendrons may include an inert, elongated polymer including a first end coupled to the dendritic core and a second end. In some examples, the dendrons and the dendritic core are disposed within a corresponding one of the wells. In some examples, the dendrons and the dendritic core substantially fill a corresponding one of the wells. In some examples, the dendrons are covalently bonded to the corresponding one of the wells. In some examples, the polynucleotide is covalently bonded to the dendritic core.

In some examples, the wells are covalently bonded to the molecules.

In some examples, the molecules are held within the wells using a non-covalent force.

In some examples, each of the molecules includes a protein to which the single respective polynucleotide is coupled. In some examples, the protein includes an antibody. In some examples, the single respective polynucleotide is coupled to an antigen for which the antibody is selective.

In some examples, the polynucleotide extends outside of the respective well.

In some examples, the polynucleotide is single-stranded.

In some examples, each of the molecules has a hydrodynamic diameter which is about 60% to about 100% of a diameter of the respective well.

In some examples, the wells have a diameter between about 10 nm and about 200 nm.

In some examples, wherein the molecules have a diameter between about 10 nm and about 200 nm.

In some examples, a pitch of the wells is at least five times a length of the single-stranded polynucleotides.

In some examples, each particle further includes a hydrogel to which the plurality of amplification primers is coupled.

In some examples, the polynucleotide includes an amplification adapter that is hybridized to the one of the amplification primers.

Some examples herein provide a method of amplifying polynucleotides. The method may include flowing a plurality of molecules into a flowcell including wells. Each molecule may include a single respective polynucleotide. The method may include coupling at least some of the wells to a single respective one of the molecules such that a single respective polynucleotide is coupled to those wells. The method may include flowing a plurality of particles into the flowcell, each particle including amplification primers. The method may include, at each well, hybridizing an amplification primer of one of the particles to the respective polynucleotide which is coupled to that well. The method may include extending that amplification primer to generate a first amplicon of the respective polynucleotide which is coupled to that well. The method may include using the amplification primers of that particle to generate amplicons of the first amplicon.

In some examples, each of the molecules includes: a dendritic core to which the single respective polynucleotide is coupled; and a plurality of dendrons. Each of the dendrons may include an inert, elongated polymer including a first end coupled to the dendritic core and a second end. In some examples, the method further includes disposing the dendrons and the dendritic core within a corresponding one of the wells. In some examples, the dendrons and the dendritic core substantially fill a corresponding one of the wells. In some examples, the method further includes covalently bonding the dendrons to the corresponding one of the wells. In some examples, the method further includes covalently bonding the polynucleotide to the dendritic core. In some examples, covalently bonding the polynucleotide to the dendritic core includes: contacting a precursor of the dendritic molecule with a template polynucleotide, wherein the precursor of the dendritic molecule includes a capture primer covalently coupled to the dendritic core, and wherein the template polynucleotide includes an adapter; hybridizing the adapter to the capture primer; extending the capture primer using the template polynucleotide to form a duplex; and dehybridizing the template polynucleotide from the duplex to leave the polynucleotide covalently coupled to the dendritic core.

In some examples, the method includes covalently bonding the wells to the molecules.

In some examples, the molecules are held within the wells using a non-covalent force.

In some examples, each of the molecules includes a protein to which the single respective polynucleotide is coupled. In some examples, the protein includes an antibody. In some examples, the single respective polynucleotide is coupled to an antigen for which the antibody is selective.

In some examples, the polynucleotide extends outside of the respective well.

In some examples, the polynucleotide is single-stranded.

In some examples, each of the molecules has a hydrodynamic diameter which is about 60% to about 100% of a diameter of the respective well.

In some examples, the wells have a diameter between about 10 nm and about 200 nm.

In some examples, the molecules have a diameter between about 10 nm and about 200 nm.

In some examples, a pitch of the wells is at least five times a length of the single-stranded polynucleotides.

In some examples, each particle further includes a hydrogel to which the plurality of amplification primers is coupled.

In some examples, the polynucleotide includes an amplification adapter that hybridizes to the one of the amplification primers.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1D schematically illustrate example operations for capturing a polynucleotide using a molecule.

FIGS. 2A-2F schematically illustrate example operations for capturing a polynucleotide using a particle and the molecule of FIG. 1D.

FIGS. 3A-3E schematically illustrate example operations for amplifying and sequencing a polynucleotide using the particle of FIG. 2F.

FIG. 4 schematically illustrates a device including a plurality of wells in which operations such as described with reference to FIGS. 2A-2F are implemented.

FIG. 5 schematically illustrates a device including a plurality of wells in which operations such as described with reference to FIGS. 3A-3D are implemented.

FIG. 6 schematically illustrates operations for capturing a polynucleotide using a particle and a molecule.

FIG. 7 schematically illustrates example operations for amplifying and sequencing a polynucleotide using the particle of FIG. 6.

FIGS. 8A-8B schematically illustrate example methods of preparing a dendritic molecule.

FIGS. 9A-9F schematically illustrate example dendritic cores that may be used in the example methods of FIGS. 8A-8B.

FIGS. 10A-10I schematically illustrate additional example dendritic cores that may be used in the example methods of FIGS. 8A-8B.

FIGS. 11A-11C schematically illustrate additional example dendritic cores that may be used in the example methods of FIGS. 8A-8B.

FIG. 12 schematically illustrates a method that was used to prepare dendritic molecules.

FIG. 13 illustrates a dynamic light scattering (DLS) trace from the dendritic molecules prepared in accordance with FIG. 12.

FIG. 14A illustrates reactions in a method with which a functionalized polymer was prepared.

FIG. 14B is a photograph of the functionalized polymer prepared in accordance with FIG. 14A.

FIG. 14C is a nuclear magnetic resonance (NMR) trace of the functionalized polymer prepared in accordance with FIG. 14A.

FIG. 14D is a gel permeation chromatography (GPC) trace of the functionalized polymer prepared in accordance with FIG. 14A, in water.

FIG. 15 schematically illustrates the way a dendritic particle prepared in accordance with FIG. 12 was used to capture a polynucleotide.

FIGS. 16A-16C schematically illustrate example operations for capturing a polynucleotide using a molecule.

### DETAILED DESCRIPTION

Examples provided herein are related to capturing and amplifying polynucleotides using particles.

Monoclonality of a cluster in a given region of a sequencing flow cell is important to obtaining a sufficient signal to noise ratio (SNR) from that region to make a base call. The particles herein may be used to generate substantially monoclonal clusters through a deterministic approach. More specifically, molecules, each of which includes a single target polynucleotide, are coupled to respective wells in a flowcell such that a single molecule (and thus a single polynucleotide) is coupled to each well. Accordingly, once coupled to a well, the molecules may sterically exclude additional molecules from being coupled to the well. Particles including amplification primers are then flowed over the wells, and are captured at respective wells when the polynucleotide at that well hybridizes to one of the amplification primers. The spacing between the wells is such that each particle may be captured at only a single one of the wells, and accordingly may hybridize to only one of the polynucleotides. The primer is extended to generate an amplicon of the target polynucleotide that is coupled to the particle. The other amplification primers of that particle then are used to generate additional amplicons, i.e., a substantially monoclonal cluster, coupled to the particle. The amplicons may be sequenced in that flowcell, or the particle may be released and then disposed on a different flowcell for sequencing of the amplicons.

First, some terms used herein will be briefly explained. Then, some example structures and example methods for capturing and amplifying polynucleotides using particles and molecules will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes have polynucleotide strands that disassociate from one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase. Exemplary polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” is defined as a polynucleotide to which nucleotides may be added via a free 3′ OH group. A primer may include a 3′ block preventing polymerization until the block is removed. A primer may include a modification at the 5′ terminus to allow a coupling reaction or to couple the primer to another moiety. A primer may include one or more moieties which may be cleaved under suitable conditions, such as UV light, chemistry, enzyme, or the like. The primer length may be any suitable number of bases long and may include any suitable combination of natural and non-natural nucleotides. A target polynucleotide may include an “adapter” that hybridizes to (has a sequence that is complementary to) a primer, and may be amplified so as to generate a complementary copy polynucleotide by adding nucleotides to the free 3′ OH group of the primer.

A “capture primer” is intended to mean a primer that is coupled to a particle and may hybridize to an adapter of a target polynucleotide. In some cases, a capture primer that is coupled to the particle and may hybridize to another adapter of that target polynucleotide may be referred to as an “orthogonal capture primer.” The adapters may have respective sequences that are complementary to those of capture primers to which they may hybridize. A capture primer and an orthogonal capture primer may have different and independent sequences than one another. A capture primer that may be used to hybridize to an adapter of a target polynucleotide in order to couple that polynucleotide to the particle, but that may not be used to grow a complementary strand during an amplification process, may in some cases be referred to as a “seeding primer.” A capture primer that may be used to grow a complementary strand during an amplification process may in some cases be referred to as an “amplification primer.”

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates may include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates may include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials may include, but are not limited to gallium arsenide, indium phosphide, aluminum, ceramics, polyimide, quartz, resins, polymers and copolymers. In some examples, the substrate and/or the substrate surface may be, or include, quartz. In some other examples, the substrate and/or the substrate surface may be, or include, semiconductor, such as GaAs or ITO. The foregoing lists are intended to be illustrative of, but not limiting to the present application. Substrates may comprise a single material or a plurality of different materials. Substrates may be composites or laminates. In some examples, the substrate comprises an organo-silicate material. Substrates may be flat, round, spherical, rod-shaped, or any other suitable shape. Substrates may be rigid or flexible. In some examples, a substrate is a bead or a flow cell.

In some examples, a substrate is or includes a particle. As used herein, a “particle” is a small localized object which exists as a discrete unit in a given medium. In detail, the term refers to microscopic particles with sizes ranging from atoms to molecules, such as a nanoparticle or colloidal particle. A particle may refer to a substrate that is sufficiently small that it may be located within a fluid (such as a liquid), that is, substantially surrounded on all sides by the fluid. As such, a particle may be carried by (moved by) the fluid in which it is located, for example by flowing the fluid from one location to another location. As explained elsewhere herein, particles can have a variety of shapes. When a particle is at least partially spherical, it may be referred to as a “bead.” In some examples, a first substrate (such as a flat or patterned surface within a flow cell) may be used to support a second substrate (such as a particle). In some examples, the largest dimension of a particle may be about 1 nm to about 2 μm, e.g., about 100 nm to about 500 nm. In some examples, the particle may be substantially spherical, in which case the largest dimension of the particle may be its diameter. However, particles may have any suitable shape, such as oval, rod, dumb-bell, or the like. Non-limiting examples of materials that are suitable for use in the present particles include biological materials such as protein, polymer, silica, magnetic materials, metal, and the like, or any suitable combination of two or more materials. In some examples, the present particles have a magnetic or super-magnetic core, which may facilitate their manipulation.

In some examples, a substrate includes a patterned surface. A “patterned surface” refers to an arrangement of different regions in or on an exposed layer of a substrate. For example, one or more of the regions may be features where one or more capture primers are present. The features can be separated by interstitial regions where capture primers are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, substrate includes an array of wells (depressions) in a surface. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of features (e.g., microwells or nanowells) on glass, silicon, plastic or other suitable material(s) with a patterned, covalently-linked hydrogel such as poly(N-(5-azidoacetamidylpentyl) acrylamide-co-acrylamide) (PAZAM). The process creates hydrogel regions used for sequencing that may be stable over sequencing runs with a large number of cycles. The covalent linking of the hydrogel to the wells may be helpful for maintaining the hydrogel in the structured features throughout the lifetime of the structured substrate during a variety of uses. However in many examples, the hydrogel need not be fully or even partially covalently linked to the wells. For example, in some conditions silane free acrylamide (SFA) may be used as the hydrogel material.

In particular examples, a structured substrate may be made by patterning a suitable material with wells (e.g. microwells or nanowells), coating the patterned material with a hydrogel material (e.g., PAZAM, SFA or chemically modified variant thereof, such as the azidolyzed version of SFA (azido-SFA)) and polishing the surface of the hydrogel coated material, for example via chemical or mechanical polishing, thereby retaining hydrogel in the wells but removing or inactivating substantially all of the hydrogel from the interstitial regions on the surface of the structured substrate between the wells.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An exemplary patterned substrate having these characteristics is the etched substrate used in connection with BEAD ARRAY technology (Illumina, Inc., San Diego, Calif.).

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that may be used in methods and compositions set forth herein include, but are not limited to, those commercially available from Illumina, Inc. (San Diego, CA).

As used herein, a “hydrogel” refers to a three-dimensional polymer network structure that includes polymer chains and is at least partially hydrophilic and contains water within spaces between the polymer chains. A hydrogel may include any suitable combination of hydrophilic, hydrophobic, and/or amphiphilic polymer(s), so long as the overall polymer network is hydrophilic and contains water within spaces between the polymer chains. Hydrogels include chemical hydrogels in which both the bonding to form the polymer chains, and any cross-linking between the polymer chains, is covalent; such cross-linking during hydrogel formation may be irreversible, as distinguished from the present reversible cross-linking which is performed after the hydrogel is formed. In some cases, the chemical hydrogel may include, or may consist essentially of, brush-like structures of polymer chains attached to a surface, substantially without physical or covalent crosslinks between polymer chains, or alternatively polymer chains with multiple attachment points to a surface, resulting in loops, but also lacking interchain crosslinks. Hydrogels also include physical hydrogels in which the bonding to form the polymer chains, and any cross-linking within the polymer chains, is not covalent. Nonlimiting examples of physical hydrogels include agarose and alginate.

As used herein, the “polymer chain” of a hydrogel is intended to mean those portions of the hydrogel that are polymerized with one another during the polymerization process. Polymer chains may be cross-linked to form the hydrogel. For example, cross-linkers may be added during or after the polymerization process that forms the polymer chains. Additionally, or alternatively, in some examples the polymer chains may be deposited on a substrate surface or particle that includes functional groups to which functional groups of the polymer chains become coupled. The polymer chains may be coupled to the surface, e.g., via reactions between the functional groups of the polymer chains and the functional groups at the surface, and such coupling may cross-link the polymer chains to form the hydrogel. Such cross-linking may cause the polymer chains to covalently or non-covalently attach to one another, or may occur as a result of chain entanglement during polymerization and/or attachment to a surface.

As used herein, the term “directly” when used in reference to a layer covering the surface of a substrate or a particle is intended to mean that the layer covers the substrate's or particle's surface without a significant intermediate layer, such as, e.g., an adhesive layer or a polymer layer. Layers directly covering a surface may be attached to this surface through any chemical or physical interaction, including covalent bonds or non-covalent adhesion.

As used herein, the term “immobilized” when used in reference to a polynucleotide is intended to mean direct or indirect attachment to a substrate or particle via covalent or non-covalent bond(s). In certain examples, covalent attachment may be used, or any other suitable attachment in which the polynucleotides remain stationary or attached to a substrate or particle under conditions in which it is intended to use the substrate or particle, for example, in polynucleotide amplification or sequencing. Polynucleotides to be used as capture primers or as target polynucleotides may be immobilized such that a 3′-end is available for enzymatic extension and at least a portion of the sequence is capable of hybridizing to a complementary sequence. Immobilization may occur via hybridization to a surface attached oligonucleotide, in which case the immobilized oligonucleotide or polynucleotide may be in the 3′-5′ orientation. Alternatively, immobilization may occur by means other than base-pairing hybridization, such as covalent attachment.

As used herein, the term “array” refers to a population of substrate regions that may be differentiated from each other according to relative location. Different particles and/or different molecules (such as polynucleotides) that are at different regions of an array may be differentiated from each other according to the locations of the regions in the array. Illustratively, an individual region of an array may include one or more molecules of a particular type. For example, a substrate region may include a single particle which is coupled to a single target polynucleotide having a particular sequence, or may be coupled to a plurality of amplicons having the same sequence as one another. The regions of an array respectively may include different features than one another on the same substrate. Exemplary features include without limitation, wells in a substrate, particles in or on a substrate, projections from a substrate, ridges on a substrate or channels in a substrate. The regions of an array respectively may include different regions on different substrates than each other.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above exemplary ranges. Exemplary polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “double-stranded,” when used in reference to a polynucleotide, is intended to mean that all or substantially all of the nucleotides in the polynucleotide are hydrogen bonded to respective nucleotides in a complementary polynucleotide.

As used herein, the term “single-stranded,” when used in reference to a polynucleotide, means that essentially none of the nucleotides in the polynucleotide are hydrogen bonded to a respective nucleotide in a complementary polynucleotide. A polynucleotide that has an “inability” to hybridize to another polynucleotide may be single-stranded, though not all single-stranded polynucleotides are unable to hybridize to certain other polynucleotides.

As used herein, the term “target polynucleotide” is intended to mean a polynucleotide that is the object of an analysis or action. The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an adapter that functions as a primer binding site, that flank(s) a target polynucleotide sequence that is to be analyzed. A target polynucleotide hybridized to a capture primer may include nucleotides that extend beyond the 5′ or 3′ end of the capture oligonucleotide in such a way that not all of the target polynucleotide is amenable to extension. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “amplicon,” when used in reference to a polynucleotide, is intended to means a product of copying the polynucleotide, wherein the product has a nucleotide sequence that is substantially the same as, or is substantially complementary to, at least a portion of the nucleotide sequence of the polynucleotide. “Amplification” and “amplifying” refer to the process of making an amplicon of a polynucleotide. A first amplicon of a target polynucleotide may be a complementary copy. Additional amplicons are copies that are created, after generation of the first amplicon, from the target polynucleotide or from the first amplicon. A subsequent amplicon may have a sequence that is substantially complementary to the target polynucleotide or is substantially identical to the target polynucleotide. It will be understood that a small number of mutations (e.g., due to amplification artifacts) of a polynucleotide may occur when generating an amplicon of that polynucleotide.

A substrate region or particle that includes substantially only amplicons of a given polynucleotide may be referred to as “monoclonal,” while a substrate region or particle that includes amplicons of polynucleotides having different sequences than one another may be referred to as “polyclonal.” A substrate region or particle that includes a sufficient number of amplicons of a given polynucleotide to be used to sequence that polynucleotide maybe referred to as “functionally monoclonal.” Illustratively a substrate region or particle in which about 60% or greater of the amplicons are of a given polynucleotide may be considered to be “functionally monoclonal.” Additionally, or alternatively, a substrate region or particle from which about 60% or more of a signal is from amplicons of a given polynucleotide may be considered to be “functionally monoclonal.” A polyclonal region of a substrate or particle may include different subregions therein that respectively are monoclonal. Each such monoclonal region, whether within a larger polyclonal region or on its own, may correspond to a “cluster” generated from a “seed.” The “seed” may refer to a single target polynucleotide, while the “cluster” may refer to a collection of amplicons of that target polynucleotide.

As used herein, a “dendritic molecule” is intended to refer to a molecule in which at least some of the atoms are arranged in multiple branches, or “dendrons,” which extend from a central region, or “dendritic core” (which may be referred to herein as a “core” for simplicity). A molecule including such features may be understood to be “dendritic,” even if not specifically referred to as a “dendritic molecule.” The core of a dendritic molecule may be branched. In some examples, a core of a dendritic molecule may include a “dendritic polyamide” which is intended to refer to a branched structure including amide bonds and to which elongated polymers may be coupled so as to form dendrons. A nonlimiting example of a dendritic polyamide is a “dendritic polypeptide,” which is intended to refer to a branched polypeptide to which elongated polymers may be coupled so as to form dendrons. In some examples, a capture primer (such as a seeding primer or amplification primer) may be coupled to the dendritic core and may be used to capture a polynucleotide having an adapter which is substantially complementary to the capture primer of the dendritic molecule.

### Structures and Methods for Capturing and Amplifying Polynucleotides Using Particles

The present particles and molecules may be used together to facilitate generation of substantially monoclonal clusters. Among other things, the present molecules may be used to capture a single target polynucleotide (e.g., only one single-stranded polynucleotide).

Additionally, each of the present particles may include amplification primers for use in capturing the polynucleotide from the molecule, and then amplifying the polynucleotide. The molecule may have a diameter which substantially matches the size of a well into which the molecule respectively becomes disposed, such that the molecule sterically inhibits any other molecule from being disposed within that well. As such, the single captured target polynucleotide may be located within the well. The polynucleotide then may be used to capture a single particle at the well, e.g., via hybridization of an amplification adapter of the polynucleotide to an amplification primer coupled to the particle. The amplification primer then may be extended to generate an amplicon of the polynucleotide that is coupled to the particle. The amplicon then may be amplified so as to generate a substantially monoclonal cluster which is coupled to the particle.

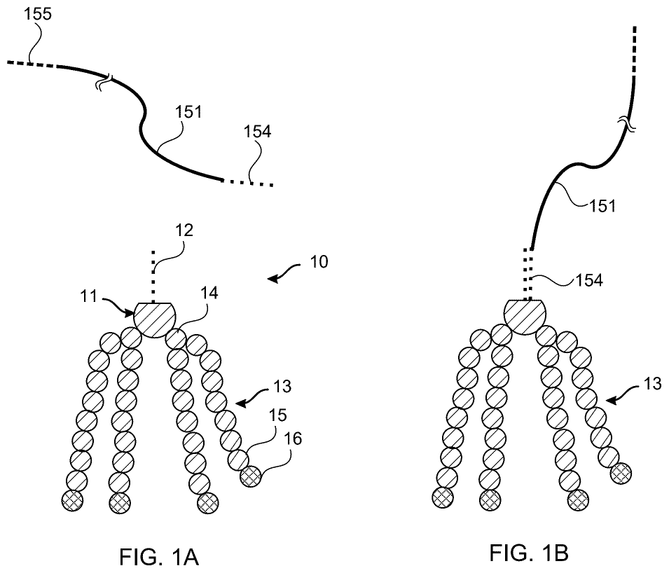

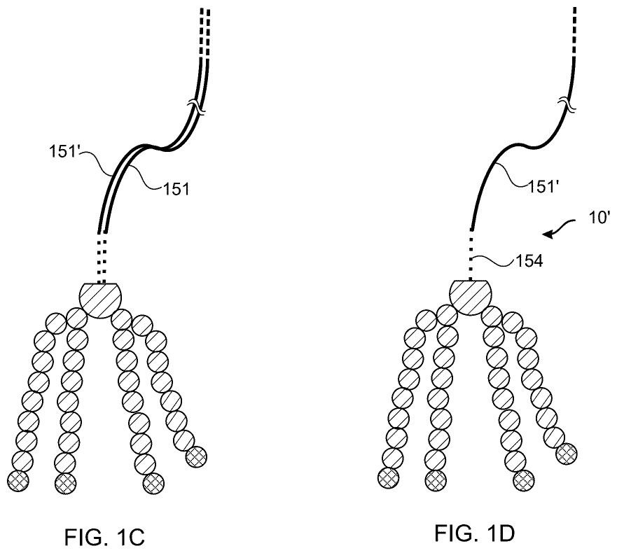

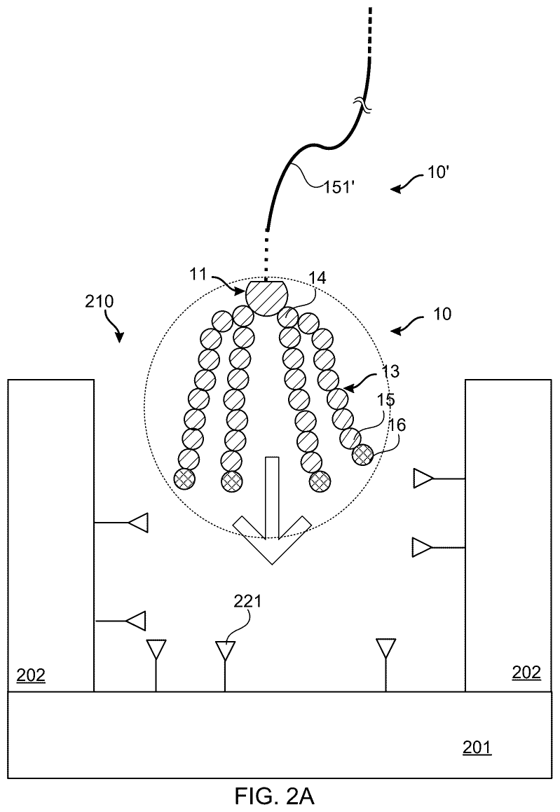

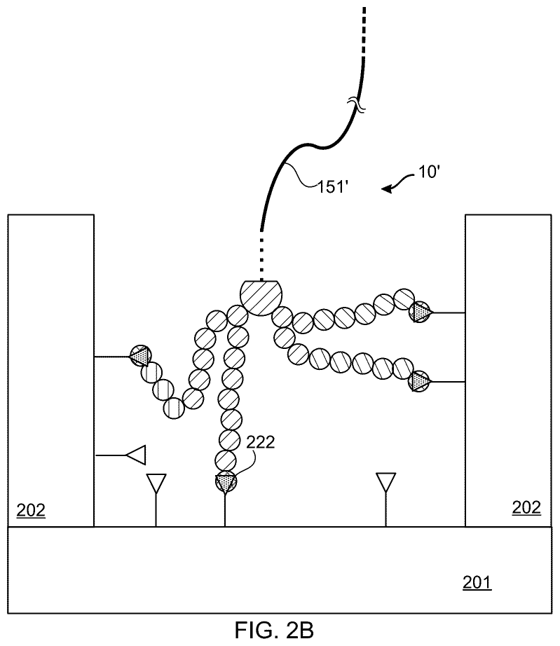

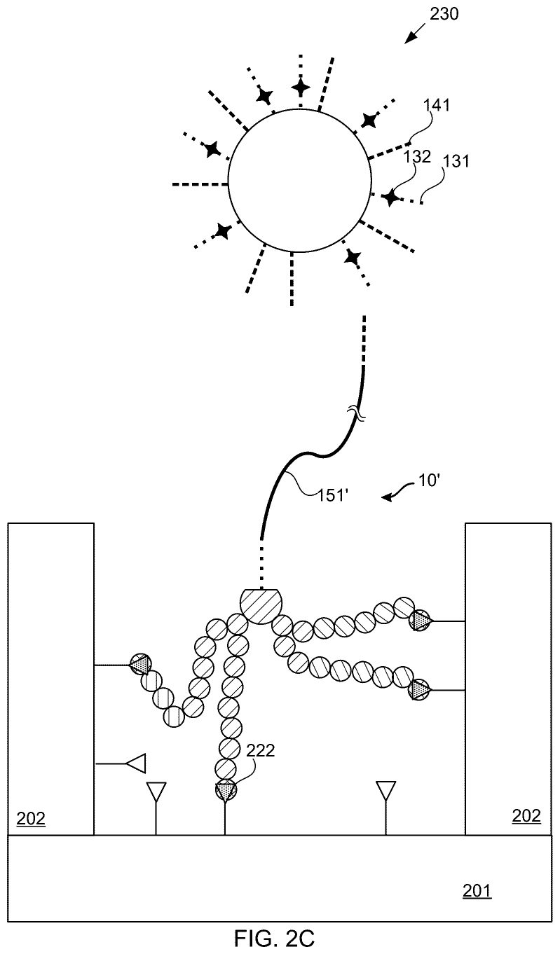

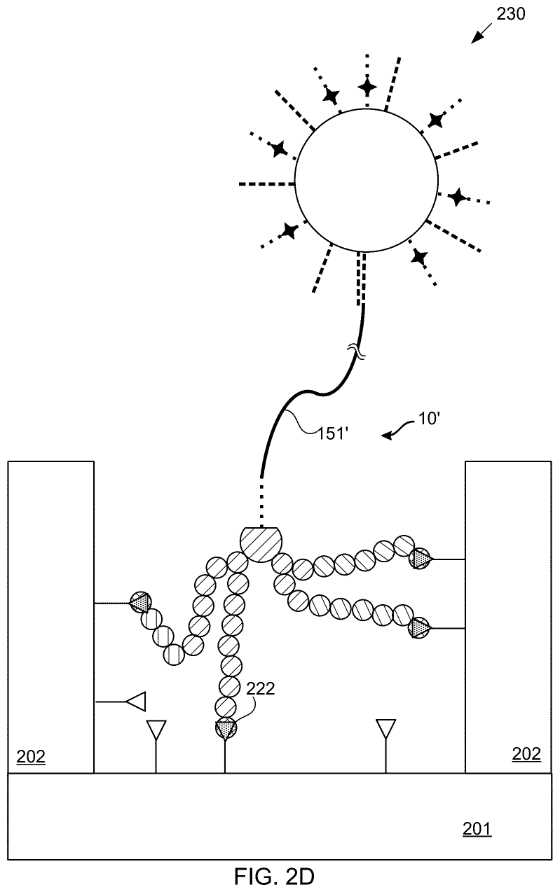

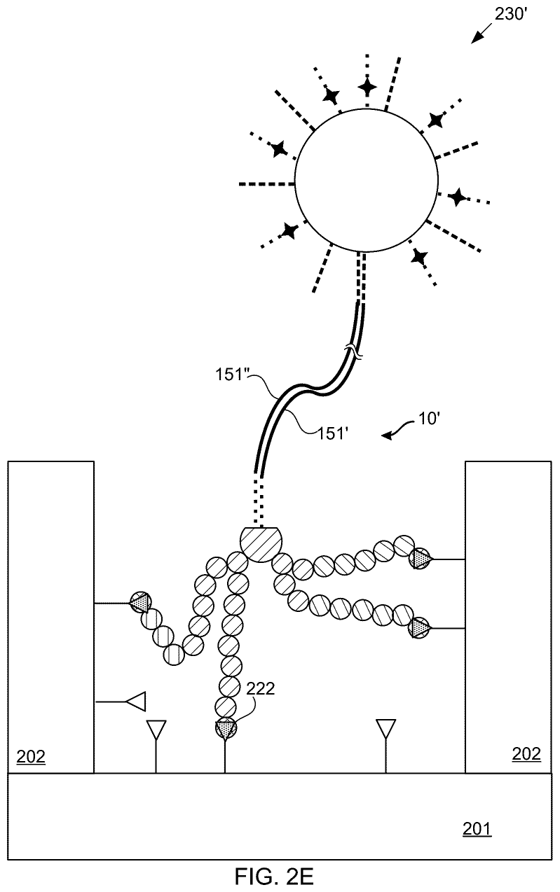

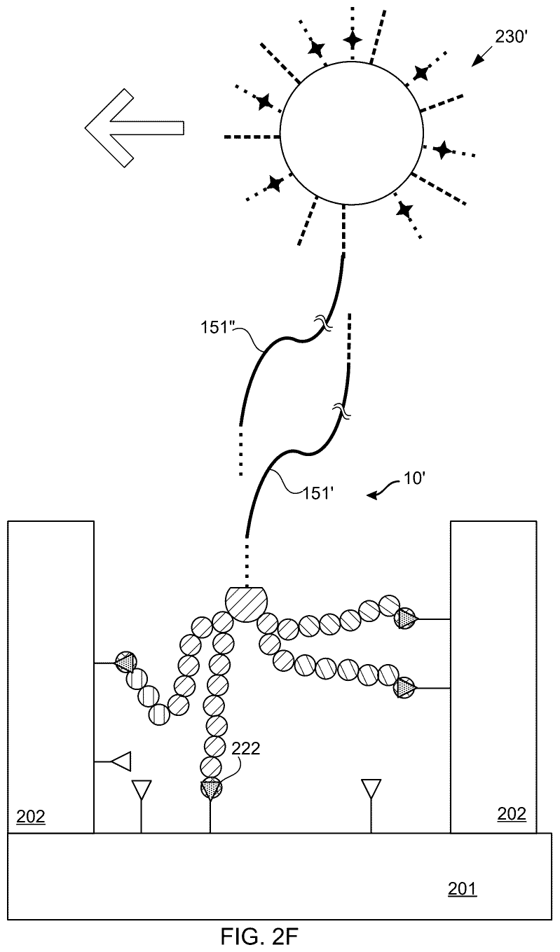

FIGS. 1A-1B schematically illustrate example operations for capturing a polynucleotide using a molecule, e.g., a dendritic molecule. Referring first to FIG. 1A, dendritic molecule 10 may include dendritic core 11; seeding primer 12 coupled to the dendritic core; and a plurality of dendrons 13. Each of dendrons 13 includes an inert, elongated polymer that includes a first end 14 coupled to the dendritic core 11 and a second end 15 coupled to a functional group 16. In some examples, the functional 16 group is to react with another functional group to form a covalent bond, e.g., in a manner such as will be described with reference to FIGS. 2A-2F. In other examples, functional group 16 may be omitted.

As illustrated in FIG. 1A, molecule 10 (e.g., a dendritic molecule) may be contacted with a fluid which may include target polynucleotides having a variety of lengths, e.g., polynucleotide fragments that are generated using commercially available fragmentation or tagmentation techniques, using DNA or RNA that it is desired to sequence. Illustratively, the molecule may be contacted with a fluid including polynucleotide 151. Although only one polynucleotide is illustrated in FIG. 1A, it will be appreciated that the fluid may include thousands, or even millions, of fragments. In some examples, the polynucleotides within the fluid may be substantially single-stranded. For example, polynucleotide 151 may not be hybridized to a complementary strand. Polynucleotide 151 may include first and second adapters, e.g., first adapter 154 and second adapter 155.

In some examples, in a manner such as illustrated in FIG. 1B, adapter 154 of polynucleotide 151 may be hybridized to the seeding primer 12 of molecule 10. In some examples, seeding primer 12 may have the sequence P5 or P7, and adapter 154 may have the complementary sequence cP5 or cP7 as appropriate. However, it will be appreciated that seeding primer 12 and adapter 154 may have any suitable sequences which are sufficiently complementary to one another to hybridize to one another. Such hybridization may be performed while molecule 10 is in solution, e.g., suspended in the same fluid as is polynucleotide 150. As illustrated in FIG. 1C, adapter 154 may be extended (e.g., using a polymerase and nucleotides, not specifically illustrated) to form an amplicon 151′ of polynucleotide 151. The amplicon 151′ may be covalently bonded to molecule 10 via that adapter 154. As illustrated in FIG. 1D, the original polynucleotide 151 then may be dehybridized, leaving a single-stranded amplicon 151′ coupled to the modified molecule 10′. However, it will be appreciated that a polynucleotide may be coupled to molecule 10 in any suitable manner, e.g., by coupling the polynucleotide to a moiety that reacts with a moiety on molecule to form a covalent or non-covalent bond.

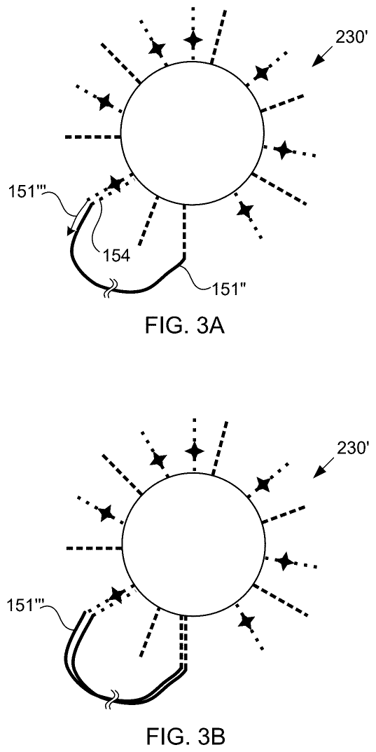

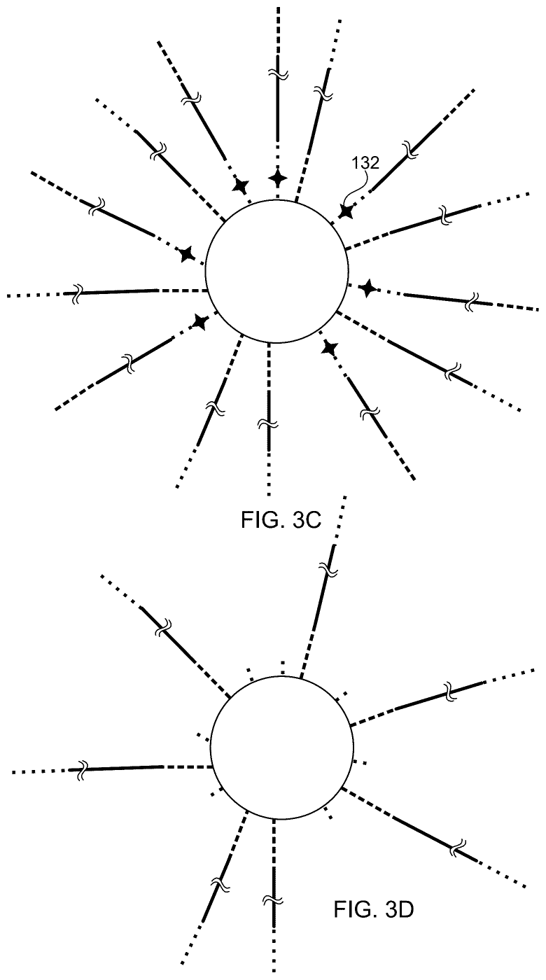

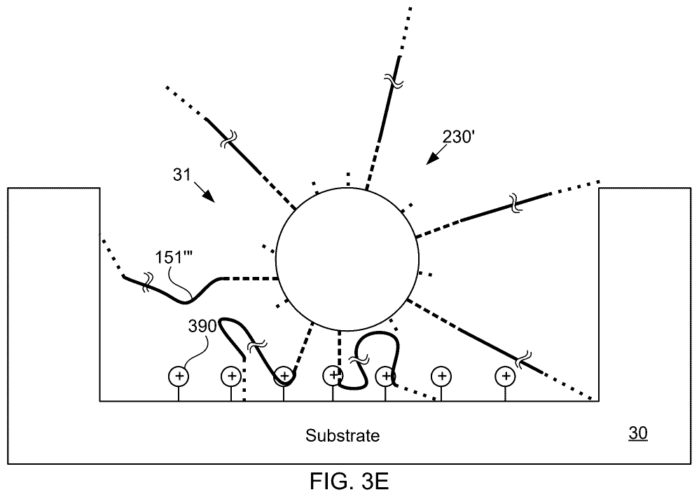

In a manner which will now be described with reference to FIGS. 2A-2F, amplicon 151′ or other polynucleotide may be captured using a particle, and then may be amplified in a manner such as described with reference to FIGS. 3A-3E to generate a substantially monoclonal cluster suitable for use in sequencing in a manner such as described with reference to FIG. 3F.

FIGS. 2A-2F schematically illustrate example operations for capturing a polynucleotide using the molecule 10′ of FIG. 1D, e.g., a molecule that is covalently bonded to a single respective polynucleotide 151′. A plurality of molecules 10′ may be flowed into a flowcell that includes a plurality of wells 210, each well including a plurality of functional groups 221. For example, the molecule 10′ which is illustrated in FIG. 2A may be one of a plurality of molecules that are suspended in a fluid. In a manner such as described with reference to FIGS. 1A-1D, each molecule 10′ of the plurality may include a dendritic core 11; a polynucleotide 151′ coupled to the dendritic core; and a plurality of dendrons 13, each of the dendrons including an elongated polymer including a first end 14 coupled to the dendritic core and a second end 15 optionally including a functional group 16.

The fluid containing the molecules 10′ may be flowed into a flowcell that includes well 210 which is one of a plurality of wells within substrate 200. Wells 210 may include a volume which is bounded, for example, by a bottom substrate 201 and one or more vertical sidewalls 202. The volume may be generated in any suitable manner, for example using nanoimprint lithography, conventional photolithography techniques, or the like. In the nonlimiting example illustrated in FIG. 2A, functional groups 221 respectively may be coupled both to bottom substrate 201 and vertical sidewall(s) 202, although it will be appreciated that functional groups may be omitted from bottom substrate 201 or from vertical sidewall(s) 202.

At least some of the wells 210 may be coupled to a single respective one of molecules 10′ such that a single respective polynucleotide 151′ is coupled to those wells. For example, within at least some of the wells 210, a respective one of the molecules 10′ may become disposed within that well. For example, as intended to be represented in FIG. 2A by the large downward-pointing arrow, molecule 10′ may become partially inserted into well 210. For example, diffusion may transport the molecule 10′ to well 210, and then interactions between the molecule and the well 210 may retain the molecule within the well. In various examples, the dendrons of dendritic molecule 10′ may covalently, non-covalently, and/or electrostatically interact with well 210. Additionally, as intended to be illustrated within FIG. 2A, molecule 10′ may have a hydrodynamic diameter denoted by the dotted circle, which sterically inhibits other molecules from entering the same well 210. As such, well 210 (and other wells within the flowcell) may receive substantially a single one of the molecules 10′. For example, each of the molecules 10′ may have a hydrodynamic diameter which is about 60% to about 100% of a diameter of wells 210. In some examples, the dendrons 13 and dendritic core 11 substantially fill a corresponding one of wells 210. Polynucleotide 151′ may extend outside of the respective well 210.

As illustrated in FIG. 2B, within wells 210 containing a single one of the molecules 10′, covalent bonds 222 are formed between the functional groups 221 of that well and the functional groups 16 of that molecule. In FIG. 2B, covalent bonds 222 are intended to be represented by the shading of the triangles representing functional groups 221, indicating that the functional groups 221 have reacted. The covalent bonds may immobilize the molecule 10′ within well 210, such that the molecule may remain in the well during subsequent operations which now will be described. As such, the polynucleotide 151′ may be retained within that well, reducing the risk that the polynucleotide inadvertently may seed an undesired area. In examples in which functional groups 16 are omitted, functional groups 221 similarly may be omitted and non-covalent binding may be used to retain molecule 10′ within well 210.

In some examples, after the molecule 10′ becomes coupled to the well 210, a plurality of particles are flowed into the flowcell. The particles may include amplification primers. In the nonlimiting example illustrated in FIG. 2C, particles 230 may be coupled to a mixture of amplification primers 131 and amplification primers 141. Amplification primers 131 and 141 may have any suitable sequences, e.g., that are orthogonal to one another. That is, amplification primer 141 may be referred to as an orthogonal amplification primer, while amplification primer 131 may be referred to as an amplification primer; or amplification primer 131 may be referred to as an orthogonal amplification primer, while amplification primer 141 may be referred to as an amplification primer. In one nonlimiting example, amplification primers 131 may include P5, and amplification primers 141 may include P7. In another nonlimiting example, amplification primers 141 may include P5, and amplification primers 131 may include P7. Optionally, particle 230 may include a hydrogel (not specifically illustrated in FIG. 2C) to which the amplification primers are coupled.

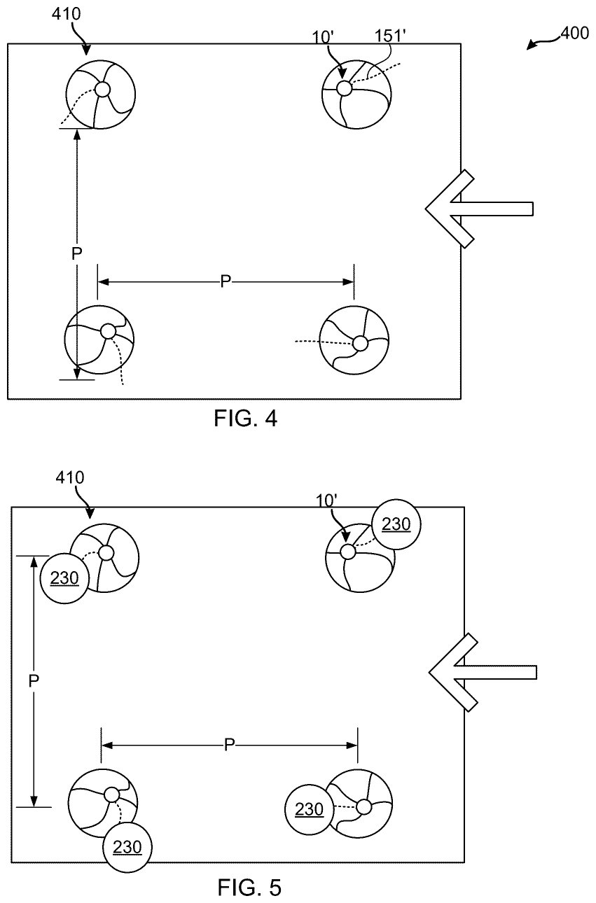

As illustrated in FIG. 2D, at each well 210, an amplification primer of one of the particles 230 may hybridize to the respective polynucleotide 151′ which is coupled to that well (via molecule 10′). For example, seeding adapter 154 of polynucleotide 151′ may hybridize to an amplification primer 131 of particle 230, or alternatively seeding adapter 155 of polynucleotide 151′ may hybridize to amplification primer 141 of particle 230. Accordingly, polynucleotide 151′ may be considered to capture particle 230. In a manner such as described with reference to FIGS. 4 and 5, a pitch of wells 210 may be selected such that each particle 230 may be captured by only a single polynucleotide 151′. For example, the wells 210 may be spaced far enough apart that once a particle becomes coupled to a first well via a first polynucleotide, it is unable to move sufficiently close to a second well to become hybridized to a second polynucleotide.

As illustrated in FIG. 2E, amplification primer 131 (or, equivalently, amplification primer 141) may be extended (e.g., using a polymerase and nucleotides, not specifically illustrated) to form an amplicon 151″ of polynucleotide 151′. The amplicon 151″ may be covalently bonded to particle 230 via that primer 131 or 141. As illustrated in FIG. 2F, polynucleotide 151′ then may be dehybridized, leaving a single-stranded amplicon 151″ coupled to modified particle 230′. In a manner which will be described with reference to FIGS. 3A-3F, amplicon 151″ may be amplified to generate a substantially monoclonal cluster suitable for use in sequencing.

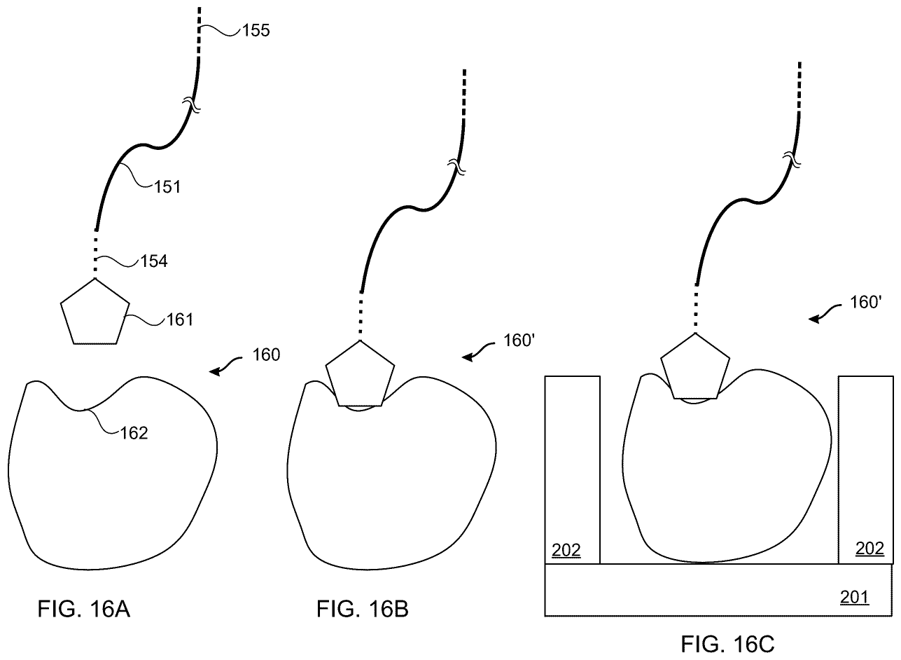

It will be appreciated that although FIGS. 1A-1D and 2A-2F illustrate a nonlimiting example in which molecules 10 are held within the wells using covalent bonds, the molecules instead may be held within the wells using a non-covalent force (e.g., in examples in which the functional group 16 is omitted, or in examples such as will be described with reference to FIGS. 16A-16C). Furthermore, although FIGS. 1A-1D and 2A-2F illustrate a nonlimiting example in which molecules 10 are dendritic, it will be appreciated that any suitable type of molecule may be used. For example, in other examples, each of the molecules may include a protein to which the single respective polynucleotide is coupled. For example, FIGS. 16A-16C schematically illustrate example operations for capturing a polynucleotide using a molecule. In the nonlimiting example illustrated in FIG. 16A, molecule 160 may include a protein that is sized to fit within wells in a similar manner as described with reference to molecule 10. As also illustrated in FIG. 16A, molecule 160 (e.g., a protein) may be contacted with a fluid which may include target polynucleotides having a variety of lengths, e.g., polynucleotide 151 including first and second adapters, e.g., first adapter 154 and second adapter 155. Polynucleotide may be coupled to ligand 161 that is couplable to site 162 of protein 160. Illustratively, the protein 160 may be or include an antibody, and the single respective polynucleotide 151 may be coupled to an antigen 161 to which the antibody becomes coupled. In another example, the protein 160 may be or include lactase, and the single respective polynucleotide 151 may be coupled to lactose 161. In another example, the protein 160 may be or include an insulin receptor, and the single respective polynucleotide 151 may be coupled to insulin 161. In another example, the protein 160 may be or include amylase, and the single respective polynucleotide 151 may be coupled to a carbohydrate 161. In some examples, ligand 161 is or includes a protein. In this regard, note that which element is considered to be a “protein” and which element is considered to be a “ligand” is arbitrary. Additional examples of protein 160 and protein ligand 161 are described in Table 1 below, as well as in the following reference, from which Table 1 is adapted: Yu et al., “Inferring high-confidence human protein-protein interactions,” BMC Bioinformatics 13, Article number: 79 (2012), the entire contents of which are incorporated by reference herein.

TABLE 1

| Protein 160 or | | Protein ligand | |
|---|---|---|---|
| protein ligand | | 161 or protein | |
| 161 | | 160 | |
| Symbol | Name | Symbol | Name |
| MDM2 | Mouse double minute 2 | TP53 | Tumor protein p53 |
| | homolog | | |
| TP53 | Tumor protein p53 | UBC | Ubiquitin C |
| HBA2 | Hemoglobin, alpha 2 | HBB | Hemoglobin, beta |
| CBL | Cas-Br-M ecotropic | EGFR | Epidermal growth factor |
| | sequence | | receptor |
| CBL | Cas-Br-M ecotropic | GRB2 | Growth factor receptor |
| | sequence | | bound 2 |
| FANCA | Fanconi anemia, | FANCG | Fanconi anemia, |
| | complementation A | | complementation G |
| EGFR | Epidermal growth factor | UBC | Ubiquitin C |
| | receptor | | |
| BRCA2 | Breast cancer 2, early onset | RAD51 | DNA repair protein |
| | | | RAD51 homolog 1 |
| HIF1A | Hypoxia inducible factor 1, | VHL | von Hippel-Lindau tumor |
| | alpha | | suppressor |
| HRAS | Ha-Ras1 proto-oncoprotein | RAF1 | Proto-oncogene c-RAF |
| SNAP25 | Synaptosomal-associated | STX1A | Syntaxin 1A |
| | protein, 25 kDa | | |
| MAX | MYC associated factor X | MYC | Proto-oncogene c-Myc |
| BARD1 | BRCA1 assoc. RING | BRCA1 | Breast cancer 1, early |
| | domain 1 | | onset |
| GRB2 | Growth factor receptor | SHC1 | SHC transforming protein |
| | bound 2 | | 1 |
| CDH1 | Cadherin 1, type 1 | CTNNB1 | Catenin, beta 1 |
| E2F1 | E2F transcription factor 1 | RB1 | Retinoblastoma 1 |
| GRB2 | Growth factor receptor | SOS1 | Son of sevenless homolog |
| | bound 2 | | 1 |
| CCNA2 | Cyclin A2 | CDK2 | Cyclin-dependent kinase 2 |
| EGF | Epidermal growth factor | EGFR | Epidermal growth factor |
| | | | receptor |
| NFKBIA | Nucl. factor of kappa light | RELA | V-rel reticulo- |
| | chain gene enhancer in B- | | endotheliosis viral |
| | cells | | oncogene homolog A |

While non-limiting examples are provided herein, it will be appreciated that any other suitable protein-ligand pair may be used to couple polynucleotide 151 to molecule 160 to form modified molecule 160′ in a manner such as illustrated in FIG. 16B. The molecule 160′ then may be disposed within a well in a manner such as illustrated in FIG. 16C, and used in a manner such as described with reference to FIGS. 2C-2F. The size of the well may be selected according to the protein which is used, such that the presence of a protein within the well inhibits another protein from entering the well.

FIGS. 3A-3E schematically illustrate example operations for amplifying and sequencing a polynucleotide using the particle 230′ of FIG. 2F. As illustrated in FIG. 3A, amplicon 151″ may bend such that adapter 154 hybridizes with one of primers 131 to form a duplex, and primer 131 may be extended to form amplicon 151′″ as illustrated in FIG. 3B. Amplicons 151″ and 151′″ repeatedly may be further amplified using bridge amplification. For example, it may be seen that the composition of FIG. 3C includes a plurality of amplicons. Amplification operations may be performed any suitable number of times so as to substantially fill the particle with an at least functionally monoclonal cluster, and in some examples a substantially monoclonal cluster, e.g., with amplicons of target polynucleotide 151 which was originally captured using molecule 10.

For example, amplicons coupled to particle 230′ may include at least about 60% amplicons of one selected target polynucleotide, or at least about 70% amplicons of one selected target polynucleotide, or at least about 80% amplicons of one selected target polynucleotide, or at least about 90% amplicons of one selected target polynucleotide, or at least about 95% amplicons of one selected target polynucleotide, or at least about 98% amplicons of one selected target polynucleotide, or at least about 99% amplicons of one selected target polynucleotide, or about 100% amplicons of one selected target polynucleotide. If amplification operations are repeated until the particle is substantially full, both adapters of the resulting amplicons may not necessarily be hybridized to corresponding capture primers or orthogonal capture primers, and as such the amplicons may extend linearly away from the particle 230 as illustrated in FIG. 3C.

In some examples, certain capture primers and orthogonal capture primers may include non-nucleotide moieties. Such non-nucleotide moieties may include, but are not limited to, excision moieties via which a portion of the capture primers selectively may be removed. For example, capture primers 131 optionally may include excision moieties 132 and/or capture primers 141 optionally may include excision moieties (excision moieties of capture primers 141 not specifically shown in FIGS. 3A-3C). The excision moieties 132 of capture primers 131 may be of the same type as, or a different type than, the excision moieties of capture primers 141. The excision moieties may be located at any suitable position along the length of any suitable primer(s) and may be, but need not necessarily be, the same type of excision moiety as one another. Following a desired number of amplification operations such as described with reference to FIGS. 3A-3C, portions of capture primers 131 may be removed by reacting a suitable enzyme or reagent with the respective excision moieties, and/or portions of capture primers 141 may be removed by reacting a suitable enzyme or reagent with the respective excision moieties. The enzyme or reagent used with the excision moieties of capture primers 131 may be the same as, or different than, the enzyme or reagent used with excision moieties of capture primers 141. Excision moieties 132 may be used to remove amplicons that are oriented in a selected direction, so that the remaining amplicons are oriented substantially in the same direction as one another in a manner such as illustrated in FIG. 3D.

In some examples, following the seeding and amplification operations, particle 230′ with amplicons coupled thereto may be disposed within a flowcell and the amplicons sequenced, e.g., using sequencing-by-synthesis. Illustratively, as shown in FIG. 3E, a substrate 30 (e.g., a surface of a flowcell) may include a plurality of positively charged moieties 390. In the nonlimiting example illustrated in FIG. 1H, the substrate 30 includes a recess 31 in which the positively charged moieties 390 are disposed. Optionally, the positively charged moieties may be coupled to a hydrogel (not specifically illustrated) which is located on the substrate (e.g., within the flowcell). The amplicons 151′″ may have a negative charge that is electrostatically attracted to the positively charged moieties 390. This electrostatic attraction may draw particle 230 into contact with the substrate. Additionally, this electrostatic attraction and/or other force(s) may retain particle 230 in contact with the substrate 30. The amplicons 151′″ then may be sequenced, e.g., using sequencing-by-synthesis operations that use reagents which may be flowed across the substrate 30 (sequencing operations not specifically illustrated).

FIG. 4 schematically illustrates a device including a plurality of wells in which operations such as described with reference to FIGS. 2A-2F are implemented. In the nonlimiting example illustrated in FIG. 4, device 400 includes a flowcell 410 including a plurality of wells 210 such as described with reference to FIG. 2A, and a plurality of molecules 10′ such as described with reference to FIG. 1D, e.g., including respective polynucleotides 151′. The molecules 10′ may be suspended in a fluid which is flowed into the flowcell in a manner as intended to be suggested by the large arrow, and coupled to respective wells 210. In some examples, once disposed in a respective well, the functional groups 16 of a molecule 10′ may become covalently bonded to the functional groups 221 of the well; in other examples, functional groups 16 and 221 may be omitted. As illustrated in FIG. 4, at least some of the wells (and optionally a majority of the wells, or more than 90% of the wells, or even substantially all of the wells) contain a single one of the molecules 10′. In one nonlimiting example, the wells 210 may have a diameter between about 10 nm and about 200 nm, e.g., between about 10 nm and about 100 nm, or between about 15 nm and about 50 nm. Additionally, or alternatively, the molecules may have a diameter between about 10 nm and about 200 nm, e.g., between about 10 nm and about 100 nm, or between about 15 nm and about 50 nm. In some examples, the hydrodynamic diameter of the molecules may be between about 20% and about 100% the diameter of the wells, e.g., between about 60% and about 100% the diameter of the wells, or between about 60% and about 90% the diameter of the wells. As such, a molecule 10′ that becomes coupled to a given well 210 may sterically inhibit any other molecules 10′ from becoming coupled to that well. For example materials and methods for preparing wells with diameters in any suitable range, see the following references, the entire contents of each of which are incorporated by reference herein: WO2016/075204; and US 2013/0096034. Example materials and methods for preparing molecules with diameters in any suitable range, are described elsewhere herein.

FIG. 5 schematically illustrates a device including a plurality of wells in which operations such as described with reference to FIGS. 3A-3D are implemented. The same device 400 may be used to implement the operations of FIGS. 3A-3D as is used to implement the operations of FIGS. 2A-2F. For example, as illustrated in FIG. 5, device 400 includes a flowcell 410 including a plurality of wells 210 which are coupled to respective molecules 10′, e.g., in a manner such as described with reference to FIG. 4. Additionally, device 400 may include a plurality of particles 230 such as described with reference to FIG. 2D, or a plurality of particles 230′ such as described with reference to FIG. 2E. Each particle 230 or 230′ may include amplification primers and may be coupled to a single one of the wells 210 via hybridization between an amplification primer of that particle and the polynucleotide 151′ of the molecule 10′ coupled to that well. Additionally, in some examples, the particle 230′ also may be coupled to the single one of the wells 210 via hybridization between amplicon 151″ and polynucleotide 151′. The particles 230 may be suspended in a fluid which is flowed into the flowcell in a manner as intended to be suggested by the large arrow, and coupled to respective wells 210. As illustrated in FIG. 5, at least some of the wells (and optionally a majority of the wells, or more than 90% of the wells, or even substantially all of the wells) are coupled to a single one of the particles 230 or 230′. In one nonlimiting example, a pitch (P) of the wells is at least five times a length of the single-stranded polynucleotides. Illustratively, device 400 may be configured for use with polynucleotides of a specified range of lengths, e.g., about 150 base pairs to about 1000 base pairs, or about 150 base pairs to about 500 base pairs, and the lengths of such polynucleotides may be selected using library preparation operations (not specifically illustrated). As such, the spacing or pitch of the wells 210 of device 400 may be selected such that, when the device is used with polynucleotides of the selected length, particles that become coupled to a well via one such polynucleotide are substantially unable to reach a polynucleotide which is coupled to a different well.

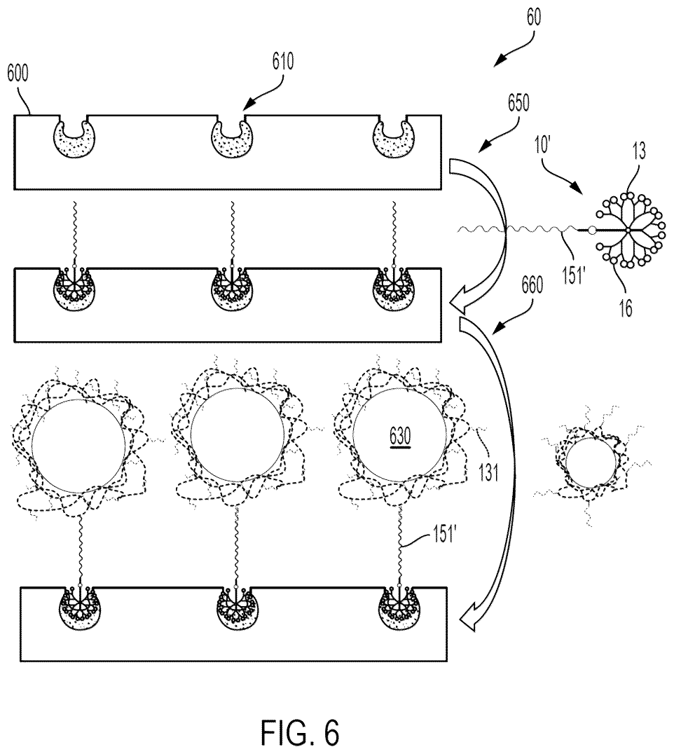

FIG. 6 schematically illustrates operations for capturing a polynucleotide using a particle and a molecule. Device 60 illustrated in FIG. 6 includes substrate 600 within which wells 610 are defined, e.g., wells with a diameter of about 15-50 nm, e.g., about 25 nm. The substrate and wells may be silanized to include functional groups. Illustratively, the substrate may include a nano-imprint lithography (NIL) resin, which may be functionalized by one or more of silanization, thiolation, surface polymerization, polymer grafting, polymer adsorption, peptide conjugation, or protein conjugation, or any combination thereof, to introduce one or more functional groups such as azide, carboxyl, NHS, amine, aldehyde, epoxy, isothiocyanate, maleimide, or the like (e.g., functional groups in Table 2). In one nonlimiting example, the substrate is silanized with norbornene silane, followed by polishing to remove the silane from the interstitials. Molecules 10′ are prepared that respectively include a target polynucleotide 151′ coupled to a dendritic molecule including dendrons 13 with functional groups 16 at the ends of the dendrons. At operation 650 illustrated in FIG. 6, molecules 620 then are flowed across device 60, and become coupled to respective ones of the wells 610 via reactions between functional groups 16 and the functional groups in the wells (illustratively, click chemistry reactions, such as azide-norbornene reactions). In some examples, the steric exclusion described herein allows only one template polynucleotide 151′ to become coupled to each well 610. At operation 660 illustrated in FIG. 6, polymer-coated and primer-grafted particles 630 are flowed over device 60, and become coupled to respective ones of the wells 610 via hybridization between polynucleotide 151′ and a primer 131 of that particle. The spacing between wells 610 may inhibit each particle from hybridizing to more than one polynucleotide, resulting in substantially monoclonal seeding of the particles 630.

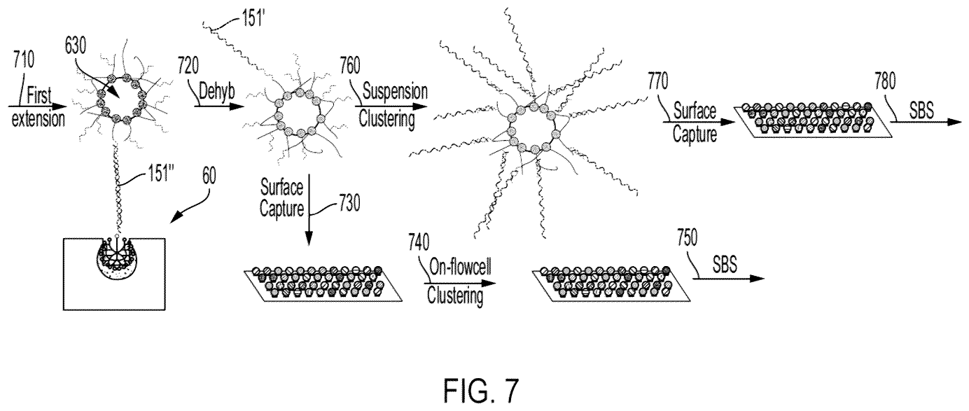

FIG. 7 schematically illustrates example operations for amplifying and sequencing a polynucleotide using the particle of FIG. 6. At operation 710 of FIG. 7, the primer may be extended in a manner such as described with reference to FIG. 2E to generate an amplicon 151″ that is coupled to the particle 630 and hybridized to polynucleotide 151′. At operation 720 of FIG. 7, the amplicon 151″ then may be dehybridized from polynucleotide 151′ to release the particle 630 from device 60. In example workflows including operation 730, particle 630 is captured at the surface of a flowcell, on-flowcell clustering (operation 740) is used to amplify amplicon 151″, and then sequencing-by-synthesis (SBS, operation 750) is performed. In example workflows including operation 760 instead of operation 730, in-suspension clustering is used to amplify amplicon 151″ (operation 760), then particle 630 is captured at the surface of a flowcell (operation 770), and then sequencing-by-synthesis (SBS, operation 780) is performed.

As noted elsewhere herein, in other examples the amplification may be performed using the same device (e.g., flowcell) at which the particles are initially captured using molecules 10′, rather than releasing the particles and transferring the particles to another device (e.g., flowcell). In some examples, the particles may remain anchored to the device using nonspecific interactions. Illustratively, the polymer or hydrogel of the bead may be anchored to an interstitial region (between the wells) of the device by heating the assembly to a temperature that sufficiently softens the device and the polymer or hydrogel that they may bond together.

Further details regarding example configurations of dendritic molecules, example materials suitable for use in dendritic molecules, and example methods of preparing dendritic molecules, now will be provided.

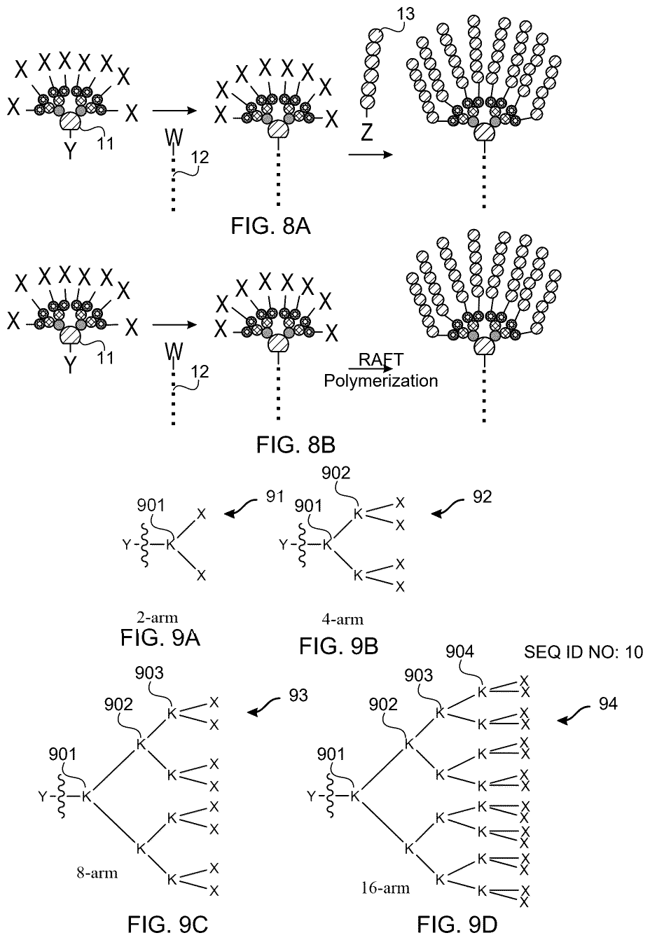

In examples in which the present molecules 10 are dendritic molecules, the molecules may include any suitable number of dendrons 13 coupled to the dendritic core 11. The dendrons and the seeding primer 12 may be coupled to the dendritic core in any suitable manner. For example, FIGS. 8A-8B schematically illustrate example methods of preparing a dendritic molecule. FIG. 8A illustrates a “grafting-to” example in which dendritic core 11 includes a functional group “Y” which may be reacted with a functional group “W” which is coupled to seeding primer 12, to couple the seeding primer to the dendritic core. In this example, dendritic core also includes a functional group “X” which may be reacted with a functional group “Z” which is coupled to an elongated polymer 13, to couple the elongated polymer to the dendritic core and thus form a dendron. Optionally, the free end of the elongated polymer 13 may include a functional group (not specifically illustrated in FIG. 8A) corresponding to functional group 16 described with reference to FIGS. 1A-1D and 2A-2F.

In some examples, seeding primer 12 and/or elongated polymer 13 illustrated in FIG. 8A respectively are attached to dendritic core 11 by covalent bonding. In some such examples, the covalent bonding is selected from the group consisting of amine-NHS ester bonding, amine-imidoester bonding, amine-pentafluorophenyl ester bonding, amine-hydroxymethyl phosphine bonding, carboxyl-carbodiimide bonding, thiol-maleimide bonding, thiol-haloacetyl bonding, thiol-pyridyl disulfide bonding, thiol-thiosulfonate bonding, thiol-vinyl sulfone bonding, aldehyde-hydrazide bonding, aldehyde-alkoxyamine bonding, hydroxy-isocyanate bonding, azide-alkyne bonding, azide-phosphine bonding, transcyclooctene-tetrazine bonding, norbornene-tetrazine bonding, azide-cyclooctyne bonding, azide-norbornene bonding, oxyamine-aldehyde bonding, SpyTag-SpyCatcher bonding, Snap-tag-O⁶-benzylguanine bonding, CLIP-tag-O²-benzylcytosine bonding, and sortase-coupling bonding.

Illustratively, dendritic core 11 may include one or more amino bonding sites, carboxy bonding sites, thiol bonding sites, alkyne bonding sites, NHS bonding sites, maleimide bonding sites, hydrazide bonding sites, alkoxyamine bonding sites, tetrazine bonding sites, aldehyde bonding sites, azido bonding sites, hydroxy bonding sites, cycloalkene bonding sites (such as transcyclooctene bonding sites or norbornene bonding sites), cycloalkyne bonding sites (such as cyclooctyne bonding sites dibenzocyclooctyne (DBCO) bonding sites, or bicyclononyne bonding sites), oxyamine bonding sites, SpyTag bonding sites, Snap-tag bonding sites, CLIP-tag bonding sites, or proteins with N-terminus recognized by sortase, or combinations thereof. In some such examples, seeding primer 12 and/or elongated polymers 13 respectively include a functional moiety that allows for covalent bonding with the corresponding site of the dendritic core. The functional moiety of the seeding primer 12 and/or elongated polymers 13 independently may include or is selected from a NHS ester moiety, an aldehyde moiety, an imidoester moiety, a pentafluorophenyl ester moiety, a hydroxymethyl phosphine moiety, a carbodiimide moiety, a maleimide moiety, a haloacetyl moiety, a pyridyl disulfide moiety, a thiosulfonate moiety, a vinyl sulfone moiety, a hydrazine moiety, an alkoxyamine moiety, an isocyanate moiety, an alkyne moiety, a cycloalkyne moiety, a phosphine moiety, a tetrazine moiety, an azido moiety, a SpyCatcher moiety, an O⁶-Benzylguanine moiety, an O⁶-Benzylcytosine moiety, or a fragment that can be subject to sortase coupling. In other examples, the functional moiety on the dendritic core and the functional moiety of the seeding primer 12 and/or elongated polymers 13 may be reversed from the specific examples provided above. Preferably, the moiety W on seeding primer 12 is of a different type different than the moiety Z on elongated polymer 13, and the dendritic core includes different types of moieties to respectively couple to seeding seeding primer 12 and to elongated polymer 13.

A non-exclusive list of complementary binding partners is presented in Table 2:

TABLE 2

| | Example moiety ″Y″ on | |
|---|---|---|
| | dendritic core 11 or moiety | Example moiety ″W″ on seeding |
| | ″W″ on seeding primer 12 or | primer 12 or moiety ″Z″ on |
| | moiety ″Z″ on elongated | elongated polymer 13 or moiety ″Y″ |
| Bonding site | polymer 13 | on dendritic core |
| amine-NHS | amine group, —NH₂ | 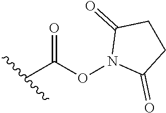 [SMILES: *C(=O)ON1C(=O)CCC1=O] |
| | | N-Hydroxysuccinimide ester |
| amine-imidoester | amine group, —NH₂ | 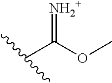 [SMILES: *C(=[NH2+])OC] |
| | | imidoester |
| amine- pentafluorophenyl ester | amine group, —NH₂ | 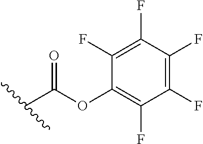 [SMILES: *C(=O)Oc1c(F)c(F)c(F)c(F)c1F] |
| | | pentafluorophenyl ester, |
| amine- hydroxymethyl phosphine | amine group, —NH₂ | 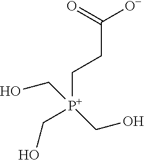 [SMILES: O=C([O-])CC[P+](CO)(CO)CO] |
| | | hydroxymethyl phosphine |
| amine-carboxylic | amine group, —NH₂ | carboxylic acid group, —C(═O)OH (e.g., |
| acid | | following activation of the carboxylic |
| | | acid by a carbodiimide such as EDC (1- |
| | | ethyl-3-(-3-dimethylaminopropyl) |
| | | carbodiimide hydrochloride) or DCC |
| | | (N′,N′-dicyclohexyl carbodiimide) to |
| | | allow for formation of an amide bond |
| | | of the activated carboxylic acid with an |
| | | amine group) |
| thiol-maleimide | thiol, —SH | 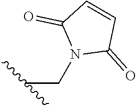 [SMILES: *CN1C(=O)C=CC1=O] |
| | | maleimide |
| thiol-haloacetyl | thiol, —SH | 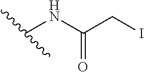 [SMILES: *NC(=O)CI] |
| | | haloacetyl (e.g., iodoacetyl or other |
| | | haloacetyl) |
| thiol-pyridyl disulfide | thiol, —SH | 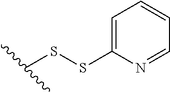 [SMILES: *SSc1ccccn1] |
| | | pyridyl disulfide |
| thiol-thiosulfonate | thiol, —SH | 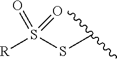 |
| | | thiosulfonate |
| thiol-vinyl sulfone | thiol, —SH | 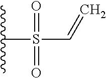 [SMILES: *S(=O)(=O)C=C] |
| | | vinyl sulfone |
| aldehyde- hydrazide | aldehyde, —C(═O)H | 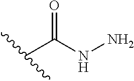 [SMILES: *C(=O)NN] |
| | | hydrazide |
| aldehyde- alkoxyamine | aldehyde, —C(═O)H | 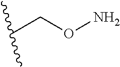 [SMILES: *CON] |
| | | alkoxyamine |
| hydroxy- isocyanate | hydroxyl, —OH | 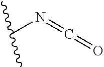 [SMILES: *N=C=O] |
| | | isocyanate |
| azide-alkyne | azide, —N₃ | 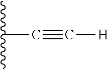 [SMILES: *C#C] |
| | | alkyne |
| azide-phosphine | azide, —N₃ | 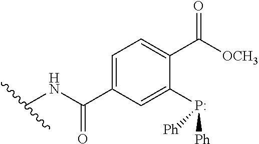 |
| | | phosphine, e.g .: |
| azide-cyclooctyne | azide, —N₃ | 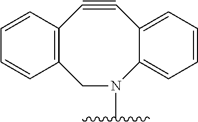 [SMILES: *N1Cc2ccccc2C#Cc2ccccc21] |
| | | cyclooctyne, e.g. dibenzocyclooctyne |
| | | (DBCO) |
| | | 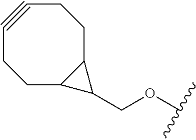 [SMILES: *OCC1C2CCC#CCCC21] |
| | | or BCN (bicyclo[6.1.0]nonyne) |
| azide-norbornene | azide, —N₃ | 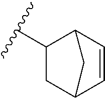 [SMILES: *C1CC2C=CC1C2] |
| | | norbornene |
| transcyclooctene- tetrazine | 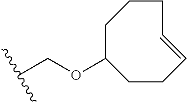 [SMILES: *COC1CC/C=C/CCC1] | 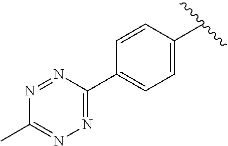 [SMILES: *c1ccc(-c2nnc(C)nn2)cc1] |
| | transcyclooctene | tetrazine, e.g., benzyl-methyltetrazine |
| norbornene- tetrazine | 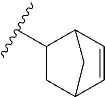 [SMILES: *C1CC2C=CC1C2] | 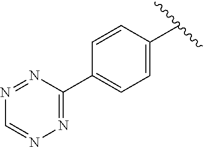 [SMILES: *c1ccc(-c2nncnn2)cc1] |
| | norbornene | tetrazine, e.g. benzyl-tetrazine |
| oxime | aldehyde or ketone (e.g., amine | alkoxyamine |
| | group or N-terminus of | |
| | polypeptide converted to an | |
| | aldehyde or ketone by pyridoxal | |
| | phosphate) | |
| SpyTag- | SpyTag: amino acid sequence | SpyCatcher amino acid sequence: |
| SpyCatcher | AHIVMVDAYKPTK (SEQ ID | MKGSSHHHHHHVDIPTTENLYFQ |
| | NO:5) | GAMVDTLSGLSSEQGQSGDMTIEE |
| | | DSATHIKFSKRDEDGKELAGATME |
| | | LRDSSGKTISTWISDGQVKDFYLY |
| | | PGKYTFVETAAPDGYEVATAITFT |
| | | VNEQGQVTVNGKATK (SEQ ID |
| | | NO:6) |
| SNAP-tag-O⁶- Benzylguanine | SNAP-tag (O-6-methylguanine- DNA methyltransferase) | 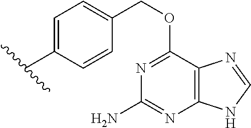 [SMILES: *c1ccc(COc2nc(N)nc3[nH]cnc23)cc1] |
| | | O⁶-Benzylguanine |
| CLIP-tag-O²- benzylcytosine | CLIP-tag (modified O-6- methylguanine-DNA methyltransferase) | 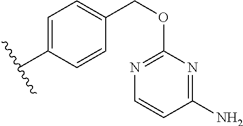 [SMILES: *c1ccc(COc2nccc(N)n2)cc1] |
| | | O²-benzylcytosine |
| Sortase-coupling | -Leu-Pro-X-Thr-Gly (SEQ ID | -Gly(3-5) (SEQ ID NO:8) (SEQ ID |
| | NO:7) | NO:9) |

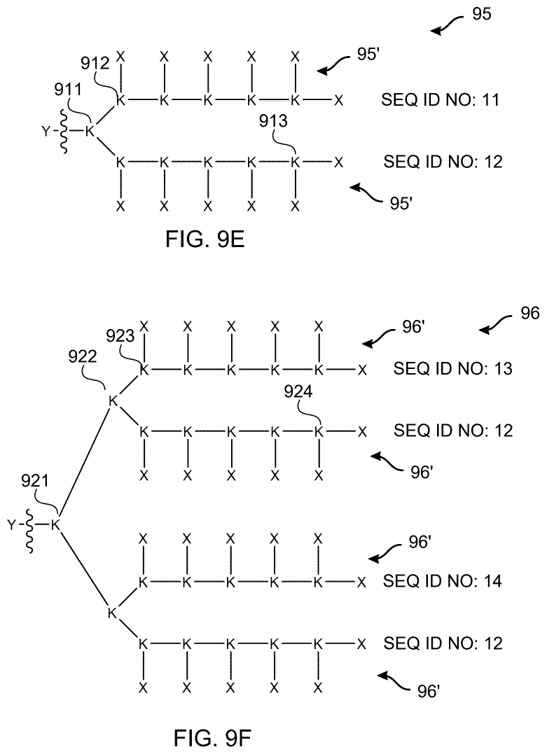

While FIG. 8A illustrates an example in which the elongated polymer is formed before being coupled to the dendritic core to form the dendrons, in other examples the elongated polymer is grown directly from the dendritic core, e.g., using RAFT polymerization or atom transfer radical polymerization (ATRP) to form the dendrons. For example, FIG. 8B illustrates an example in which the primer is coupled using a “grafting-to” approach and the dendrons are coupled using a “grafting-from” approach. More specifically, in the nonlimiting example of FIG. 81B, dendritic core 11 includes a functional group “Y” which may be reacted with a functional group “W” which is coupled to seeding primer 12, to couple the seeding primer to the dendritic core in a manner such as described with reference to FIG. 8A. In this example, dendritic core also includes a functional group “X” from which an elongated polymer 13 may be grown (e.g., using RAFT polymerization or ATRP) to form a dendron. In some examples, X is a RAFT agent (such as a dithiobenzoate, trithiocarbonate, dithiocarbamate, or xanthate) or a chain transfer agent (CTA) for RAFT, or a halogen-containing macroinitiator for ATRP. Some examples of controlled radical polymerizations that may be used to form elongated polymer 13 include, but are not limited to, RAFT, ATRP, nitroxide-mediated polymerization (NMP), catalytic chain transfer polymerization (CCTP). In examples in which the elongated polymer 13 is formed using RAFT polymerization, the polymer may include, for example, a polystyrene, polyacrylate, polyacrylamide, polymethacrylate, polymethacrylamide, polyvinyl ester, or polyvinyl amide. For further details regarding forming dendritic polymers using polymerization, see the following references, the entire contents of each of which are incorporated by reference herein: Taton et al., “Controlled polymerizations as tools for the design of star-like and dendrimer-like polymers,” Polymer International, 55(1): 1138-1145 (2006); and Gillies et al., “Dendrimers and dendritic polymers in drug delivery,” Drug Discovery Today 10(1): 35-43 (2005). In still other examples such as described with reference to FIGS. 9A-9F, elongated polymer 13 may be formed using peptide synthesis or oligosynthesis. Optionally, the free end of the elongated polymer 13 may include a functional group (not specifically illustrated in FIG. 8B) corresponding to functional group 16 described with reference to FIGS. 1A-1D and 2A-2F.

While FIGS. 8A-8B illustrate nonlimiting examples including eight functional groups “X” to which respective elongated polymers 13 may be attached, or from which respective elongated polymers 13 may be formed, it will be appreciated that the dendritic core may include any suitable number of functional groups “X,” e.g., two or more, or four or more, illustratively four, six, eight, sixteen, thirty-two, or the like. Branched molecules including desired chemical functional groups are commercially available. For example, FIGS. 9A-9F schematically illustrate example dendritic cores that may be used in the example methods of FIGS. 8A-8B. FIG. 9A illustrates an example 2-arm dendritic core 91. Dendritic core 91 includes first generation focal point 901, illustratively lysine (denoted “K”) which may be used because it has an N-terminal NH₂ and also side chain NH₂ which allows a single K to grow into 2 arms. Focal point 901 is coupled to two functional groups X and to one functional group Y. The squiggle between K and Y in FIG. 9A (and other figures herein) is intended to denote that the coupling between these two elements may not necessarily be direct; however the absence of squiggles between other elements should not be inferred to mean that the coupling between those elements must necessarily be direct.

FIG. 9B illustrates an example 4-arm dendritic core 92, including first generation focal point 901 which is coupled to two second generation focal points 902. Each of second generation focal points 902 of core 92 is coupled to two functional groups X for a total of four functional groups. Additionally, dendritic core 92 is coupled to one functional group Y. FIG. 9C illustrates an example 8-arm dendritic core 93, including first generation focal point 901 which is coupled to two second generation focal points 902. Each of second generation focal points 902 of core 93 is coupled to two third generation focal points 903. Each of third generation focal points 903 is coupled to two functional groups X for a total of eight functional groups. Additionally, dendritic core 93 is coupled to one functional group Y. FIG. 9D illustrates an example 16-arm dendritic core 94, including first generation focal point 901 which is coupled to two second generation focal points 902. Each of second generation focal points 902 of core 93 is coupled to two third generation focal points 903. Each of third generation focal points 903 is coupled to two fourth generation focal points 904, each of which is coupled to additional functional groups X for a total of sixteen functional groups. Additionally, dendritic core 94 is coupled to one functional group Y.

While FIGS. 9A-9D illustrate examples in which outer branches of the dendritic core respectively are coupled to one functional group X and in which inner branches of the dendritic core are coupled to other focal points and not to functional groups, other examples readily may be envisioned. For example, FIG. 9E illustrates an example two-arm dendritic core 95. Dendritic core 95 includes focal point 911 which is coupled to one functional group Y, and is coupled to two additional generation focal points 912. Accordingly, dendritic core 95 is branched at focal point 911. As shown in FIG. 9E, each of the additional focal points 912 is coupled to another such additional focal point, and also is coupled to a functional group X. A plurality of such additional focal points 912 are linearly coupled to one another along each of the two arms 95′, 95′ of dendritic core 95. The end of each arm may include a further focal point 912 which is coupled to two functional groups X. Accordingly, the dendritic core 95 includes a plurality of arms (e.g., two arms), each of which arms includes plurality of functional groups X along that arm.

As another example, FIG. 9F illustrates an example four-arm dendritic core 96. Dendritic core 96 includes focal point 921 which is coupled to one functional group Y, and is coupled to two additional generation focal points 922. As shown in FIG. 9F, each of the additional focal points 922 is coupled to two further focal points 923. Each further focal point 923 is coupled to another such additional focal point, and also is coupled to a functional group X. Accordingly, dendritic core 96 is branched at focal point 921 and at focal points 922. A plurality of such further focal points 923 are linearly coupled to one another along each of the two arms 96′, 96′ of dendritic core 96. The end of each arm may include an additional focal point 924 which is coupled to two functional groups X. Accordingly, the dendritic core 96 includes a plurality of arms (e.g., four arms), each of which arms includes plurality of functional groups X along that arm.

The dendritic core, and the dendrons, may have any suitable respective compositions. In various examples, the dendritic core may include a branched polypeptide or a branched polyester, ester, amide, ethylene glycol, or oligonucleotide. Additionally, or alternatively, in some examples the inert, elongated polymer may include a polypeptide, polyester, polyamide, poly(ethylene glycol), or oligonucleotide.

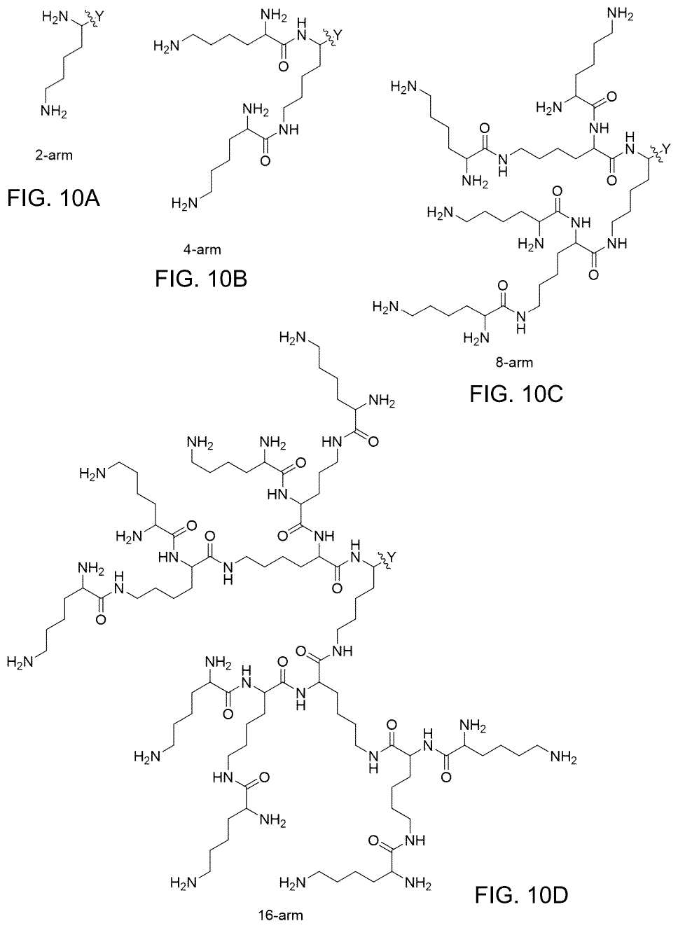

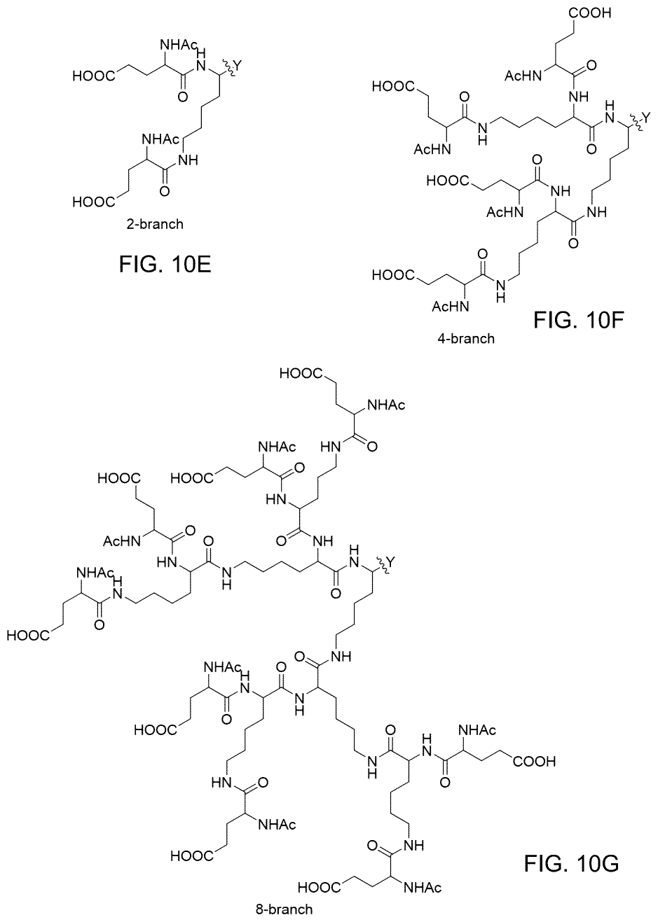

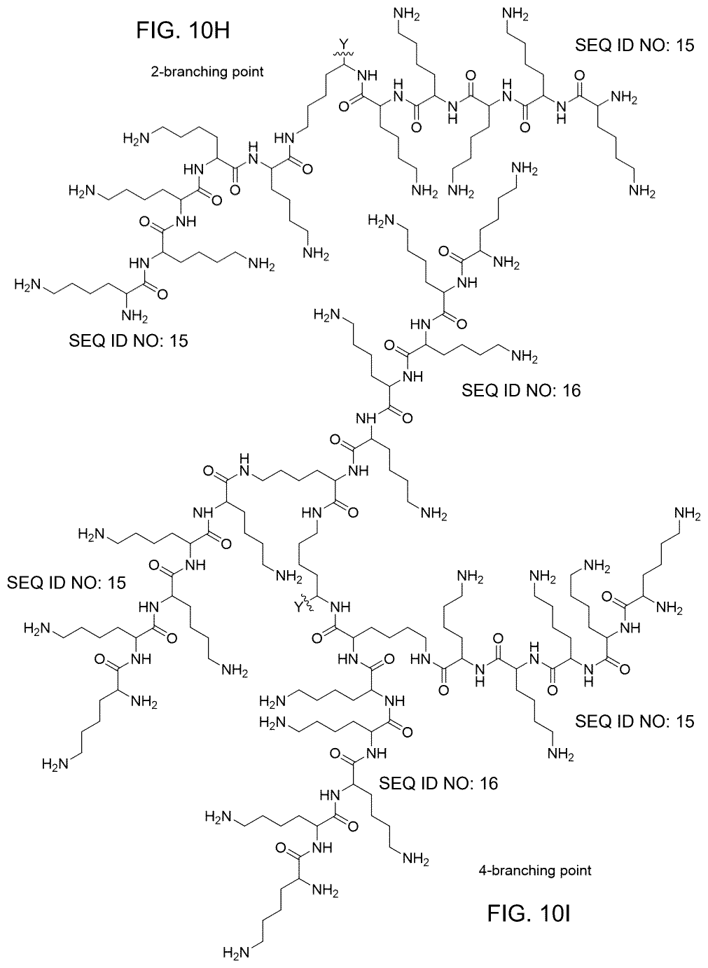

FIGS. 10A-10I schematically illustrate additional example dendritic cores that may be used in the example method of FIGS. 8A-8B. For example, FIG. 10A illustrates a nonlimiting example of a two-arm dendritic core having a configuration similar to that of FIG. 9A, and which is a branched polypeptide that includes two amine groups (—NH₂) corresponding to “X” in FIGS. 8A, and one “Y” group (such as —COOH). FIG. 10B illustrates a nonlimiting example of a four-arm dendritic core having a configuration similar to that of FIG. 9B, and which is a branched polypeptide that includes four “X” amine groups (—NH₂) and one “Y” group (such as —COOH). FIG. 10C illustrates a nonlimiting example of an eight-arm dendritic core having a configuration similar to that of FIG. 9C, and which is a branched polypeptide that includes eight “X” amine groups (—NH₂) and one “Y” group (such as —COOH). FIG. 10D illustrates a nonlimiting example of a sixteen-arm dendritic core having a configuration similar to that of FIG. 9D, and which is branched polypeptide that includes sixteen “X” amine groups (—NH₂) and one “Y” group (such as —COOH). Examples such as illustrated in FIGS. 10A-10D may be particularly suitable for use in examples such as described with reference to FIG. 8A, e.g., may be readily coupled to preformed polymer dendrons using the —NH₂ X groups, and readily may be formed using repetitive NH₂ to COOH coupling on a resin/solid support using peptide chemistry, followed by cleavage from the resin and purification to obtain the desired peptide. Traditional peptide synthesis occurs from the C→N terminal.

As another example, FIG. 10E illustrates a nonlimiting example of a two-arm dendritic core having a configuration similar to that of FIG. 9A, and which is a branched polypeptide that includes two —COOH groups corresponding to “X” in FIGS. 8A-8B and 9A-9D, and one “Y” group (such as —NH₂). FIG. 10F illustrates a nonlimiting example of a four-arm dendritic core having a configuration similar to that of FIG. 9B, and which is a branched polypeptide that includes four “X”-COOH groups and one “Y” group (such as —NH₂). FIG. 10C illustrates a nonlimiting example of an eight-arm dendritic core having a configuration similar to that of FIG. 9C, and which is a branched polypeptide that includes eight “X”-COOH groups and one “Y” group (such as —NH₂). Examples such as illustrated in FIGS. 10E-10G may be particularly suitable for use in examples such as described with reference to FIG. 8A, e.g., may be readily coupled to preformed polymer dendrons using the —COOH X groups. Examples such as illustrated in FIGS. 10E-10G also may be particularly suitable for use in examples such as described with reference to FIG. 8B. For example, a RAFT agent for use in generating dendrons using RAFT polymerization may be coupled to the —COOH X groups. Note that any amines within the branched peptide may be protected (e.g., using acyl groups, as illustrated in FIGS. 10E-10G).

As yet another example, FIG. 10H illustrates a nonlimiting example of a two-arm, two-branching point dendritic core having a configuration similar to that of FIG. 9E, and which is a branched polypeptide that includes twelve amine groups (—NH₂) corresponding to “X” in FIGS. 8A, and one “Y” group (such as —COOH). FIG. 10B illustrates a nonlimiting example of a four-arm, four-branching point dendritic core having a configuration similar to that of FIG. 9F, and which is a branched polypeptide that includes twenty-four “X” amine groups (—NH₂) and one “Y” group (such as —COOH). Examples such as illustrated in FIGS. 10A-10D may be particularly suitable for use in examples such as described with reference to FIG. 8A, e.g., may be readily coupled to preformed polymer dendrons using the —NH₂ X groups. The squiggles in FIGS. 10A-10I are intended to represent that the Y group may be coupled directly or indirectly to the rest of the dendritic molecule. For example, Y may be an amino acid residue, or Y may not be an amino acid residue and may be coupled to the rest of the dendritic molecule via an amino acid residue which is represented by the squiggle.

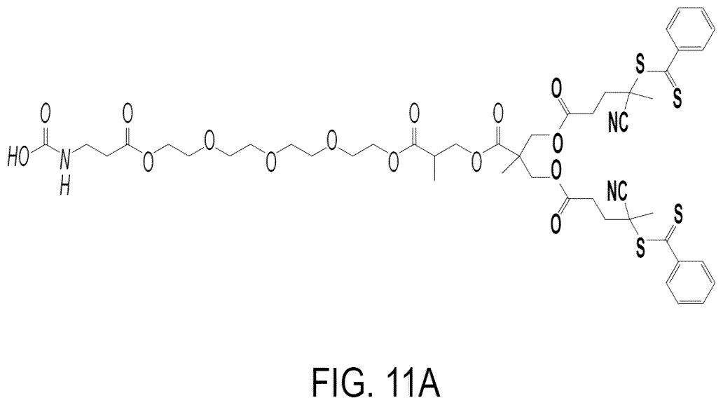

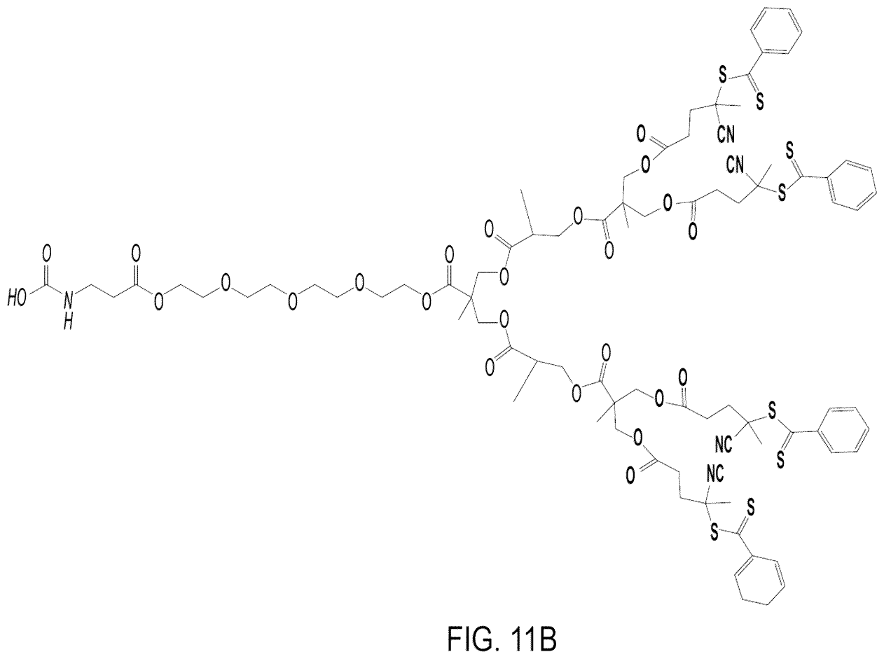

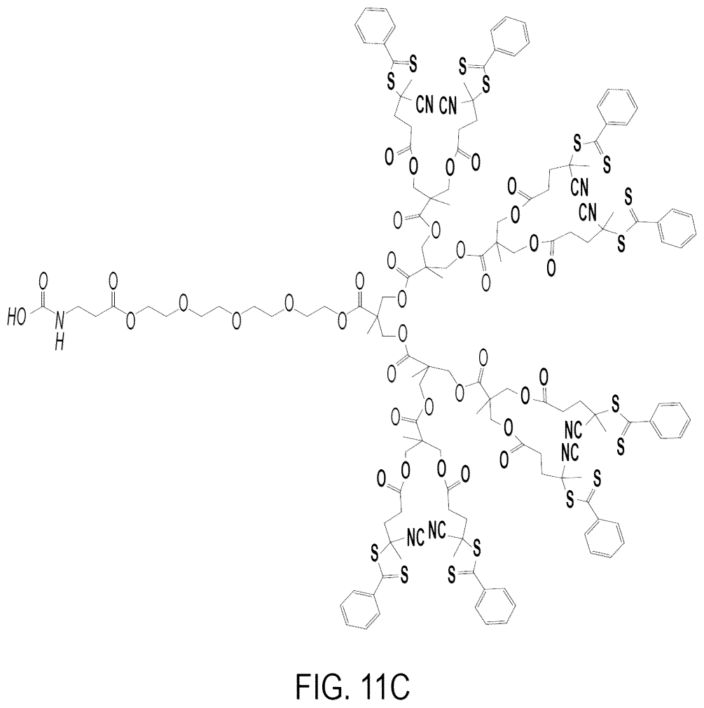

FIGS. 11A-11C schematically illustrate additional example dendritic cores that may be used in the example method of FIGS. 8A-8B. For example, FIG. 11A illustrates a nonlimiting example of a two-arm dendritic core having a configuration similar to that of FIG. 9A, and which is a branched polyester bis-MPA dendron, generation 1 including a carboxyl core, and 2 RAFT chain transfer agent (CTA) end groups. Here, CTA=4-Cyano-4-(phenylcarbonothioylthio)pentanoic acid, although another option is (methylol)propionic acid (MPA). The CTA end groups correspond to “X” in FIG. 8B. The carboxyl core corresponds to the “Y” group. Although X is CTA in the illustrated example, X may be any suitable initiator for controlled polymerization, some examples are RAFT, ATRP, NMP and CCTP. FIG. 11B illustrates a nonlimiting example of a four-arm dendritic core having a configuration similar to that of FIG. 9B, and which is a polyester bis-MPA dendron, generation 2 including a carboxyl core, four CTA end groups with CTA=4-Cyano-4-(phenylcarbonothioylthio)pentanoic acid. FIG. 11C illustrates a nonlimiting example of an eight-arm dendritic core having a configuration similar to that of FIG. 9C, and which is a polyester bis-MPA dendron, generation 3 with carboxyl core and eight CTA end groups with CTA=4-Cyano-4-(phenylcarbonothioylthio)pentanoic acid. Dendritic cores such as described with reference to FIGS. 11A-11C may be commercially available, e.g., from Polymer Factory Sweden AB (Stockholm, Sweden). Examples such as illustrated in FIGS. 11A-11C may be particularly suitable for use in examples such as described with reference to FIG. 8B.

Molecules of elongated polymer 13 illustrated in FIG. 8A may be formed in any suitable manner to include a terminal —COOH group, e.g., using a reversible addition fragmentation chain transfer (RAFT) polymerization technique. The —COOH group of respective polymer molecules 13 may be coupled to the amine groups (or other suitable groups) of the dendritic core in any suitable manner, for example using DMTMM amide-coupling agent, to form dendrons. Optionally, the polymer molecules may include a different, functional end group (such as an alkyne, amine, carboxylic acid, acetylene, N-hydroxy succinimide, biotin, or thiol) corresponding to functional group 16 described with reference to FIG. 1A. For further details regarding RAFT polymerization, including generating functional groups at the end of a polymer formed using RAFT polymerization, see Perrier, “50th anniversary perspective: RAFT polymerization—a user guide,” Macromolecules 50(19): 7433-7447 (2017), the entire contents of which are incorporated by reference herein.

From the foregoing disclosure, it will be appreciated that primers and adapters having any suitable sequences may be used. In one nonlimiting example, amplification primers 131 are P5 amplification primers, and the amplification primers 141 are P7 amplification primers. P5 amplification primers, which are commercially available from Illumina, Inc. (San Diego, CA) have the sequence 5′-AATGATACGGCGACCACCGA-3′ (SEQ ID NO: 1). P7 amplification primers, which also are commercially available from Illumina, Inc., have the sequence 5′-CAAGCAGAAGACGGCATACGA-3′ (SEQ ID NO: 2). Adapters 154 may be full-length complementary P5 adapters (cP5) having the sequence 5′-TCGGTGGTCGCCGTATCATT-3′ (SEQ ID NO: 3), and are commercially available from Illumina, Inc. Adapters 155 may be full-length complementary P7 adapters (cP7) having the sequence 5′-TCGTATGCCGTCTTCTGCTTG-3′ (SEQ ID NO: 4), and are commercially available from Illumina, Inc. Seeding primers 12 may have any suitable sequence which may hybridize with adapters 154 or may hybridize with adapters 155 (e.g., may be P5 or P7).

It will further be appreciated that various examples herein may be used with operations consistent with “bridge amplification” or “surface-bound polymerase chain reaction” and/or with other amplification modalities. One such amplification modality is “exclusion amplification,” or ExAmp. Exclusion amplification methods may allow for the amplification of a single target polynucleotide per substrate region and the production of a substantially monoclonal population of amplicons in a substrate region. For example, the rate of amplification of the first captured target polynucleotide within a substrate region may be more rapid relative to much slower rates of transport and capture of target polynucleotides at the substrate region. As such, the first target polynucleotide captured in a substrate region may be amplified rapidly and fill the entire substrate region, thus inhibiting the capture of additional target polynucleotide in the same substrate region. Alternatively, if a second target polynucleotide attaches to same substrate region after the first polynucleotide, the relatively rapid amplification of the first polynucleotide may fill enough of the substrate region to result in a signal that is sufficiently strong to perform sequencing by synthesis (e.g., the substrate region may be at least functionally monoclonal). The use of exclusion amplification may also result in super-Poisson distributions of monoclonal substrate regions; that is, the fraction of substrate regions in an array that are functionally monoclonal may exceed the fraction predicted by the Poisson distribution.

Increasing super-Poisson distributions of useful clusters is useful because more functionally monoclonal substrate regions may result in higher quality signal, and thus improved SBS; however, the seeding of target polynucleotides into substrate regions may follow a spatial Poisson distribution, where the trade-off for increasing the number of occupied substrate regions is increasing the number of polyclonal substrate regions. One method of obtaining higher super-Poisson distributions is to have seeding occur quickly, followed by a delay among the seeded target polynucleotide. The delay, termed “kinetic delay” because it is thought to arise through the biochemical reaction kinetics, gives one seeded target polynucleotide an earlier start over the other seeded targets. Exclusion amplification works by using recombinase to facilitate the invasion of primers (e.g., primers attached to a substrate region) into double-stranded DNA (e.g., a target polynucleotide) when the recombinase mediates a sequence match. The present compositions and methods may be adapted for use with recombinase to facilitate the invasion of the present capture primers and orthogonal capture primers into the present target polynucleotides when the recombinase mediates a sequence match. Indeed, the present compositions and methods may be adapted for use with any surface-based polynucleotide amplification methods such as thermal PCR, chemically denatured PCR, and enzymatically mediated methods (which may also be referred to as recombinase polymerase amplification (RPA), strand invasion, or ExAmp). For further details of amplification processes such as may be used during, and/or are compatible with, operations such as described with reference to FIGS. 2C-2D, see International Patent Application No. PCT/US2022/053005 to Ma et al., filed Dec. 15, 2022 and entitled “Hybrid Clustering,” and International Patent Application No. PCT/EP2023/058307 to Ma et al., filed Mar. 30, 2023 and entitled “Paired-End Resynthesis Using Blocked P5 Primers,” the entire contents of each of which are incorporated by reference herein.

### WORKING EXAMPLES

The following examples are intended to be purely illustrative, and not limiting of the present invention.

These examples highlight the successful synthesis of peptide-based dendritic molecules covalently bonded to a seeding primer (FIG. 1A), and the coupling of target polynucleotides to such molecules.

### Example 1. Synthesis

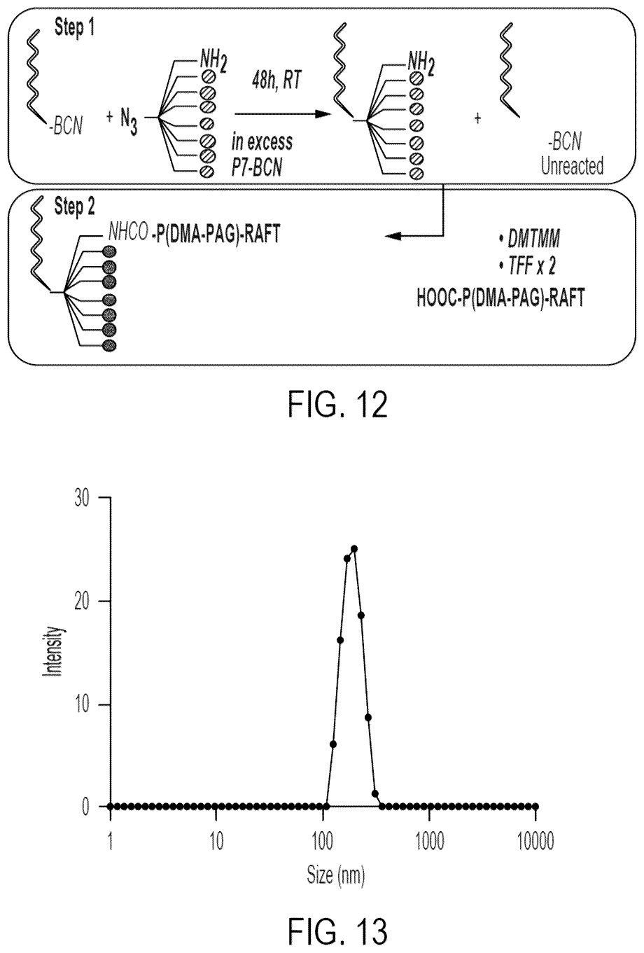

FIG. 12 schematically illustrates a method that was used to prepare dendritic molecules, and FIG. 13 illustrates a dynamic light scattering (DLS) trace from the dendritic molecules prepared in accordance with FIG. 12.

More specifically, FIG. 12 demonstrates a two-step, one-pot synthesis of an oligo-peptide dendritic molecule (OPD). In step 1, a BCN-ended P5 primer was reacted with an N₃-terminated 8-arm NH₂ dendritic core at room temperature, in accordance with the reaction between W and Y in FIG. 8. In step 2 of FIG. 12, pre-formed random copolymer-polymer molecules (dimethyl acrylamide-co-propargyl acrylate) with carboxylic acid end groups (HOOC-P(DMA-co-PAG)-RAFT) are coupled to the amine (—NH₂) end groups of the dendritic core via DMTMM coupling, in accordance with the reaction between X and Z in FIG. 8A. After purification via tangential flow filtration (TFF) a 100 kDa molecular weight cutoff (MWCO) membrane, OPDs with an average size of 200 nm were collected, see DLS trace in FIG. 13.

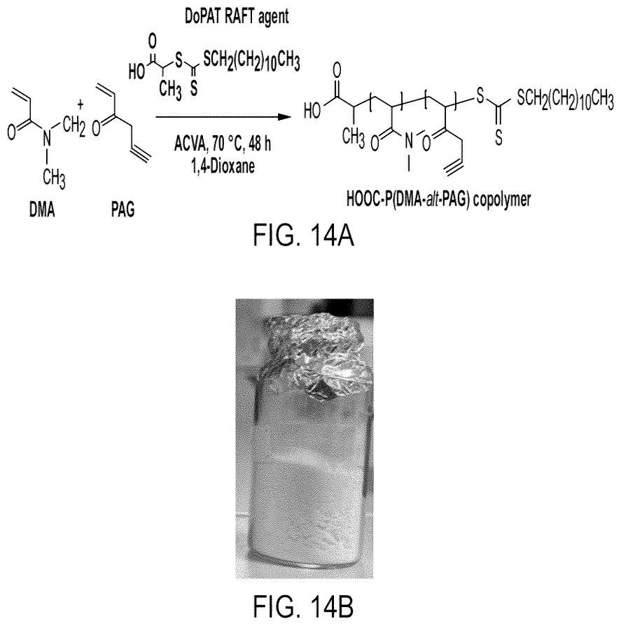

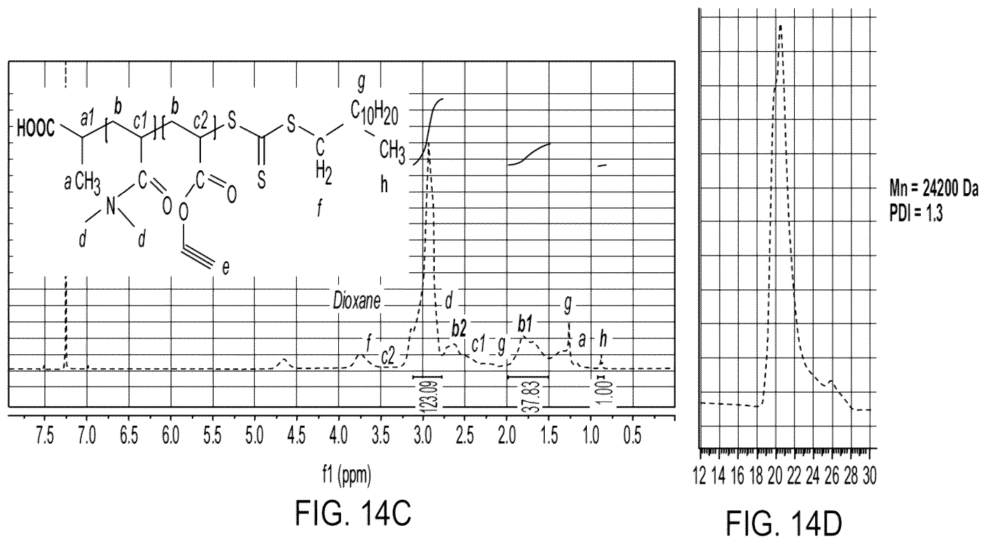

FIG. 14A illustrates reactions in a method with which a functionalized polymer was prepared. FIG. 14B is a photograph of the functionalized polymer prepared in accordance with FIG. 14A. FIG. 14C is a nuclear magnetic resonance (NMR) trace of the functionalized polymer prepared in accordance with FIG. 14A. FIG. 14D is a gel permeation chromatography (GPC) trace of the functionalized polymer prepared in accordance with FIG. 14A, in water. More specifically, the synthesis and characterization of pre-formed polymers HOOC-P(DMA-co-PAG)-RAFT which were used in Step 2 of FIG. 12 is illustrated in FIG. 14A using RAFT polymerization with DoPAT RAFT agent, which carries carboxylic acid functionality. FIG. 14B shows a photograph of the polymer powder. NMR analysis shown in FIG. 14C indicates that the degree of polymerization (DP) was approximately equal to 60. According to GPC analysis, the polymer shows a number average molecular weight of approximately 24 KDa with a PDI of 1.3 (FIG. 14D).

### Example 2. Seeding

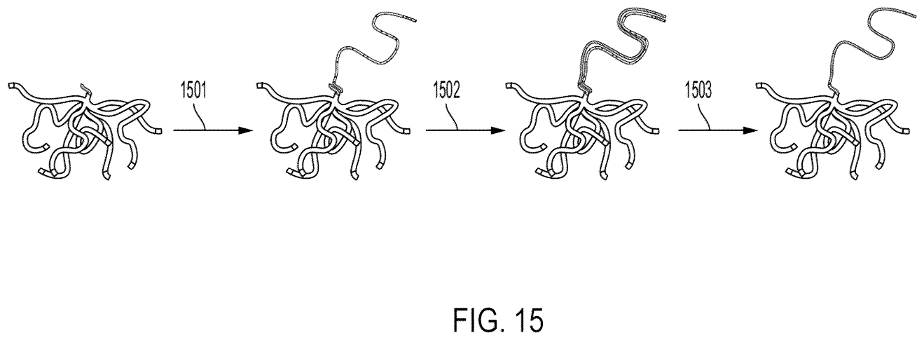

After synthesis, as described in example 1, the OPD was then seeded as described with reference to FIGS. 1A-1D). FIG. 15 schematically illustrates the way a dendritic particle prepared in accordance with FIG. 12 was used to capture and amplify a polynucleotide.

First, PhiX library polynucleotides were seeded onto respective OPDs in solution with the ratio of 1:1 or 2:1 PhiX:OPD (operation 1501). More specifically, the P5 primer attached to the dendritic core of the OPD was hybridized to the cP5 at the end of the PhiX library. DNA chain extension is then carried out for 20 min at 60° C., (operation 1502) followed by de-hybridization in NaOH (operation 1503) and TFF purification using 100 nm pore-size membrane to remove un-hybridized libraries.

### Additional Comments

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

What is claimed is:

1. A device, comprising:
a flowcell comprising wells;
a plurality of molecules, each molecule comprising a single respective polynucleotide, wherein at least some of the wells are coupled to a single respective one of the molecules such that a single respective polynucleotide is coupled to those wells; and
a plurality of particles, each particle comprising amplification primers and being coupled to a single one of the wells via hybridization between an amplification primer of that particle and the polynucleotide of the molecule coupled to that well.

2. The device of claim 1, wherein each of the molecules comprises:
a dendritic core to which the single respective polynucleotide is coupled; and
a plurality of dendrons, each of the dendrons comprising an inert, elongated polymer comprising a first end coupled to the dendritic core and a second end.

3. The device of claim 2, wherein the dendrons and the dendritic core are disposed within a corresponding one of the wells.

4. The device of claim 2 or claim 3, wherein the dendrons and the dendritic core substantially fill a corresponding one of the wells.

5. The device of any one of claims 2 to 4, wherein the dendrons are covalently bonded to the corresponding one of the wells.

6. The device of any one of claims 2 to 5, wherein the polynucleotide is covalently bonded to the dendritic core.

7. The device of any one of claims 1 to 6, wherein the wells are covalently bonded to the molecules.

8. The device of any one of claims 1 to 4 or 6, wherein the molecules are held within the wells using a non-covalent force.

9. The device of claim 1 or claim 8, wherein each of the molecules comprises a protein to which the single respective polynucleotide is coupled.

10. The device of claim 9, wherein the protein comprises an antibody.

11. The device of claim 10, wherein the single respective polynucleotide is coupled to an antigen for which the antibody is selective.

12. The device of any one of claims 1 to 11, wherein the polynucleotide extends outside of the respective well.

13. The device of any one of claims 1 to 12, wherein the polynucleotide is single-stranded.

14. The device of any one of claims 1 to 13, wherein each of the molecules has a hydrodynamic diameter which is about 60% to about 100% of a diameter of the respective well.

15. The device of any one of claims 1 to 14, wherein the wells have a diameter between about 10 nm and about 200 nm.

16. The device of any one of claims 1 to 15, wherein the molecules have a diameter between about 10 nm and about 200 nm.

17. The device of any one of claims 1 to 16, wherein a pitch of the wells is at least five times a length of the single-stranded polynucleotides.

18. The device of any one of claims 1 to 17, each particle further comprising a hydrogel to which the plurality of amplification primers is coupled.

19. The device of any one of claims 1 to 18, wherein the polynucleotide comprises an amplification adapter that is hybridized to the one of the amplification primers.

20. A method of amplifying polynucleotides, the method comprising:
flowing a plurality of molecules into a flowcell comprising wells, each molecule comprising a single respective polynucleotide;
coupling at least some of the wells to a single respective one of the molecules such that a single respective polynucleotide is coupled to those wells;
flowing a plurality of particles into the flowcell, each particle comprising amplification primers;
at each well, hybridizing an amplification primer of one of the particles to the respective polynucleotide which is coupled to that well;
extending that amplification primer to generate a first amplicon of the respective polynucleotide which is coupled to that well; and
using the amplification primers of that particle to generate amplicons of the first amplicon.

21. The method of claim 20, wherein each of the molecules comprises:
a dendritic core to which the single respective polynucleotide is coupled; and
a plurality of dendrons, each of the dendrons comprising an inert, elongated polymer comprising a first end coupled to the dendritic core and a second end.

22. The method of claim 21, comprising disposing the dendrons and the dendritic core within a corresponding one of the wells.

23. The method of claim 21 or claim 22, wherein the dendrons and the dendritic core substantially fill a corresponding one of the wells.

24. The method of any one of claims 21 to 23, further comprising covalently bonding the dendrons to the corresponding one of the wells.

25. The method of any one of claims 21 to 24, further comprising covalently bonding the polynucleotide to the dendritic core.

26. The method of claim 25, wherein covalently bonding the polynucleotide to the dendritic core comprises:
contacting a precursor of the dendritic molecule with a template polynucleotide, wherein the precursor of the dendritic molecule comprises a capture primer covalently coupled to the dendritic core, and wherein the template polynucleotide comprises an adapter;
hybridizing the adapter to the capture primer;
extending the capture primer using the template polynucleotide to form a duplex; and
dehybridizing the template polynucleotide from the duplex to leave the polynucleotide covalently coupled to the dendritic core.

27. The method of any one of claims 20 to 26, comprising covalently bonding the wells to the molecules.

28. The method of any one of claims 21 to 23, 25, or 26, wherein the molecules are held within the wells using a non-covalent force.

29. The method of claim 21 or claim 28, wherein each of the molecules comprises a protein to which the single respective polynucleotide is coupled.

30. The method of claim 29, wherein the protein comprises an antibody.

31. The method of claim 30, wherein the single respective polynucleotide is coupled to an antigen for which the antibody is selective.

32. The method of any one of claims 20 to 31, wherein the polynucleotide extends outside of the respective well.

33. The method of any one of claims 20 to 32, wherein the polynucleotide is single-stranded.

34. The method of any one of claims 20 to 33, wherein each of the molecules has a hydrodynamic diameter which is about 60% to about 100% of a diameter of the respective well.

35. The method of any one of claims 20 to 34, wherein the wells have a diameter between about 10 nm and about 200 nm.

36. The method of any one of claims 20 to 35, wherein the molecules have a diameter between about 10 nm and about 200 nm.

37. The method of any one of claims 20 to 36, wherein a pitch of the wells is at least five times a length of the single-stranded polynucleotides.

38. The method of any one of claims 20 to 37, each particle further comprising a hydrogel to which the plurality of amplification primers is coupled.

39. The method of any one of claims 20 to 38, wherein the polynucleotide comprises an amplification adapter that hybridizes to the one of the amplification primers.


SEQUENCE LISTING (16 sequences)

SEQ ID NO: 1 - DNA, 20 bp, synthetic construct
  source 1..20: mol_type = other DNA; note = P5 amplification primer
aatgatacggcgaccaccga

SEQ ID NO: 2 - DNA, 21 bp, synthetic construct
  source 1..21: mol_type = other DNA; note = P7 amplification primer
caagcagaagacggcatacga

SEQ ID NO: 3 - DNA, 20 bp, synthetic construct
  source 1..20: mol_type = other DNA; note = complementary P5 adapter
tcggtggtcgccgtatcatt

SEQ ID NO: 4 - DNA, 21 bp, synthetic construct
  source 1..21: mol_type = other DNA; note = complementary P7 adapter
tcgtatgccgtcttctgcttg

SEQ ID NO: 5 - protein, 13 aa, synthetic construct
  source 1..13: note = SpyTag: amino acid sequence
AHIVMVDAYKPTK

SEQ ID NO: 6 - protein, 134 aa, synthetic construct
  source 1..134: note = SpyCatcher amino acid sequence
MKGSSHHHHHHVDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATK

SEQ ID NO: 7 - protein, 5 aa, synthetic construct
  source 1..5: note = Sortase-coupling
LPXTG

SEQ ID NO: 8 - protein, 4 aa, synthetic construct
  source 1..4: note = Sortase-coupling
GGGG

SEQ ID NO: 9 - protein, 5 aa, synthetic construct
GGGGG

SEQ ID NO: 10 - protein, 4 aa, synthetic construct
  source 1..4: note = dendritic core
  SITE 1: note = the side chain is linked to a functional group
  SITE 2..4: note = the side chain is linked to another lysine
  SITE 1: note = the N-terminal is linked to a functional group
  SITE 4: note = the C-terminal is linked to a functional group
KKKK

SEQ ID NO: 11 - protein, 6 aa, synthetic construct
  source 1..6: note = dendritic core
  SITE 1: note = the N-terminal is linked to a functional group
  SITE 1..5: note = the side chain is linked to a functional group
  SITE 6: note = the side chain linked to another lysine
  SITE 6: note = the C-terminal is linked to a functional group
KKKKKK

SEQ ID NO: 12 - protein, 5 aa, synthetic construct
  source 1..5: note = dendritic core
  SITE 1: note = the N-terminal is linked to a functional group
  SITE 1..5: note = the side chain is linked to a functional group
  SITE 5: note = the C-terminal is linked to the side chain of another lysine
KKKKK

SEQ ID NO: 13 - protein, 7 aa, synthetic construct
  source 1..7: note = dendritic core
  SITE 1: note = the N-terminal is linked to a functional group
  SITE 1..5: note = the side chain is linked to a functional group
  SITE 6..7: note = the side chain is linked to another lysine
  SITE 7: note = the C-terminal is linked to a functional group
KKKKKKK

SEQ ID NO: 14 - protein, 6 aa, synthetic construct
  source 1..6: note = dendritic core
  SITE 1: note = the N-terminal is linked to a functional group
  SITE 1..5: note = the side chain is linked to a functional group
  SITE 6: note = the side chain is linked to another lysine
  SITE 6: note = the C-terminal is linked to the side chain of another lysine
KKKKKK

SEQ ID NO: 15 - protein, 5 aa, synthetic construct
  source 1..5: note = dendritic core
  SITE 5: note = the C-terminal is linked to a functional group
KKKKK

SEQ ID NO: 16 - protein, 6 aa, synthetic construct
  source 1..6: note = dendritic core
  SITE 6: note = the C-terminal is linked to a functional group
KKKKKK